(19) United States
(12) Patent Application Publication
(10) Pub. No.: US 2023/0348921 A1
(43) Pub. Date: Nov. 2, 2023

(54) GENE EDITING MOLECULAR CLONING KITS

(71) Applicant: Grow More Foundation, Stony Brook, NY (US)

(72) Inventors: Kate CREASEY KRAINER, Stony Brook, NY (US); Florian HAHN, Oxford (GB)

(21) Appl. No.: 18/182,936

(22) Filed: Mar. 13, 2023

Related U.S. Application Data
(60) Provisional application No. 63/319,052, filed on Mar. 11, 2022.

(51) Int. Cl. C12N 15/82; C12N 9/22; C12N 15/11

(52) U.S. Cl. CPC C12N 15/8213; C12N 9/22; C12N 15/11; C12N 2310/20; C12N 2800/80

(57) ABSTRACT

The present disclosure relates to compositions, systems, and kits for modifying a gene sequence of interest in plants and producing a gene-edited plant, part, or cell. The present disclosure further relates to methods for obtaining a gene sequence of interest to target using a CRISPR-Cas9 system, and methods for introducing such gene modification into plants.

### CROSS-REFERENCE TO RELATED APPLICATION

This application claims priority to, and the benefit of U.S. Provisional Application No. 63/319,052, filed on Mar. 11, 2022, which is hereby incorporated herein by reference in its entirety.

### FIELD

The present disclosure relates to gene or genome editing molecular technologies for producing plants with desired traits. Also, provided are kits for efficiently transforming plants with the constructs and/or vector for using a CRISPR-Cas system, and methods for modifying genes of interest in plants.

### DESCRIPTION OF THE TEXT FILE SUBMITTED ELECTRONICALLY

The Sequence Listing XML associated with this application is provided electronically in XML file format and is hereby incorporated by reference into the specification. The contents of the electronic sequence listing GRMO_002_01US_SeqList_ST26.xml; Size: 25,618 bytes; and Date of Creation: Mar. 12, 2023) are herein incorporated by reference in its entirety, and is being submitted electronically via USPTO Patent Center.

### BACKGROUND

In countries facing food insecurity, crop failure significantly not only impacts the economic livelihoods of farmers, but also can result in famine throughout the countries. The United Nations instructed governments to propose measures to achieve the 17 Sustainable Development Goals by 2030. Several of these goals relate to ending world hunger. In the developing countries, hunger and malnutrition have wide-ranging and long-lasting negative impact on physical, mental, and social health. Pests and pathogens are a constant threat to agricultural output and food production is negatively impacted by drought and flooding, exacerbated by climate change.

Traits, such as insect and disease resistance, nutrient biofortification, and drought or flood tolerance, have been successfully demonstrated in model plant species and economically important crops. However, orphan crops that aren't internationally traded lag far behind other crops such as maize, soybean, and wheat in terms of applications of advanced biotechnological methods and new breeding techniques (e.g. cutting-edge genome editing technology).

There is an unmet need for effective compositions, systems, and/or kits capable of precisely editing gene and/or genome in plants (e.g. orphan crops) in order for the plants to obtain desired traits, thereby providing established resources for orphan crop development and addressing their own agricultural needs for self-sufficiency.

### SUMMARY

The present disclosure provides a gene editing cloning system characterized by a plurality of expression cassettes. The first expression cassette comprises at least two guide RNAs (gRNAs), each of which comprises a guide sequence operably linked to a gRNA scaffold sequence. In some embodiments, a stop signal is operably linked to the gRNA scaffold sequence. The second expression cassette comprises a gene encoding a CRISPR-associated (Cas) protein and the third expression cassette comprises a selectable marker gene. In some embodiments of the system, each expression cassette comprises a promoter. In some embodiments of the system, the gene editing cloning system is utilized to edit a genome in a plant, a plant part or a plant cell thereof. In some embodiments of the system, the gRNA comprises at least 15 nucleotides guide sequence complementary to a target gene sequence. In some embodiments of the system, the gRNA scaffold sequence is a nucleic acid sequence comprising SEQ ID NO:2, or a sequence at least 90% identical thereto. In some embodiments, the gRNA scaffold sequence is a nucleic acid sequence at least 95%, 98%, or 99% identical to SEQ ID NO:2.

In some embodiments, the promoter for expression of each gRNA is a rice OsU6-2 promoter, a rice OsU3 promoter, an Arabidopsis AtU6-26 promoter, a maize ZmU3 promoter, or a soybean GmU6 promoter. In some embodiments, the promoter for expression of the gene encoding the Cas protein is a 2× 35S Cauliflower mosaic virus (CaMV) promoter. In some embodiments, the promoter for expression of the selectable marker gene is a 35S Cauliflower mosaic virus (CaMV) promoter. In some embodiments, the Cas protein is Cas9, which is plant optimized. In some embodiments, the selectable marker gene is hygromycin phosphotransferase (HPTII) gene. In some embodiments, the stop signal is poly thymine (poly T). In some embodiments, the gRNA binds to at least one genomic region of a target gene, thereby acquiring or enhancing a trait selected from the group consisting of biotic and abiotic traits, such as pest and pathogen resistance, insect and disease resistance, nutrient biofortification and bioavailability, drought or flood tolerance, and developmental traits such as yield, growth height, seed size, flowering time, and hardiness.

In some embodiments, the plant is a monocot or a dicot. In some embodiments, the plant is an orphan crop such as cassava, cowpea, plantain, millet, sweet potato, sorghum, teff, or yam.

The present disclosure provides a cloning kit comprising: (i) at least two cloning vectors for gRNAs, each of which comprises a cassette comprising a promoter operably linked to a lacZ gene and a gRNA scaffold; (ii) a vector comprising a cassette comprising a gene encoding a CRISPR-associated (Cas) protein; (iii) a vector comprising a cassette comprising a first selectable marker; (iv) a destination vector comprising a cassette comprising a second selectable marker. In some embodiments, the lacZ gene in each of said two cloning vectors is designed to be replaced with a guide sequence complementary to a target gene sequence in a plant cell by a restriction-ligation reaction. In some embodiments, each cassette has unique overhangs at 5′ and 3′ ends for orderly assembly of multiple cassettes or fragments. In some embodiments, multiple cassettes comprising said at least two cassettes from (i), said cassette from (ii), and said cassette from (iii) are assembled into a destination vector based on the unique overhangs. In some embodiments, the destination vector comprises the assembled multiple cassettes from (i), (ii), and (iii) vectors.

In some embodiments, the kit further comprises a premixed buffer, a first enzyme mix, a second enzyme mix, and at least one annealed primer pair as a control. In some embodiments of the kit, the first selectable marker gene is hygromycin phosphotransferase (HPTII) gene. n some embodiments of the kit, the second selectable marker gene is a red color selectable marker that is designed to be replaced with the assembled multiple cassettes.

The present disclosure provides a vector comprising the gene editing cloning system taught herein.

The present disclosure provides a plant, a plant part thereof, or a plant cell thereof, comprising the vector taught herein, wherein the target gene is edited and wherein plant confers a trait selected from the group consisting of pest and pathogen resistance, insect resistance, plant-disease resistance, drought tolerance, flood tolerance, nutrient biofortification and high yield. In some embodiments, a tissue culture of cells produced from the plant taught herein comprise the vector described herewith. In some embodiments, the target gene is edited in a seed produced by growing the plant taught herein. In some embodiments, a plant, or a plant part thereof, produced by growing the seed taught herein.

The present disclosure provides a method for obtaining a cloning vector for expression of at least two gRNAs, the method comprising: (a) preparing at least two gRNA primer pairs with an overhang at 5′ end. In some embodiments, each primer pair is complementary to anneal a double stranded guide sequence molecule, and 5′ end of each primer has the overhang. In embodiments, the method comprises (b) digesting with an restriction enzyme each vector comprising a promoter operably linked to a lacZ gene which is operably linked to a gRNA scaffold and a stop signal, wherein the lacZ gene is removed by the enzymatic reaction. The method further comprises (c) ligating the double stranded guide sequence molecule from (a) with the lacZ gene-depleted vector from (b) and (d) obtaining a cloning vector that comprises at least two expression cassettes, each of which comprises the promoter, the guide sequence, the gRNA scaffold, and the termination signal. In some embodiments of the method, the guide sequence at a 5′ end of each gRNA binds to a target gene. In some embodiments, the 5′ overhang is capable of ligating to BsaI restriction site. In some embodiments, the restriction enzyme is BsaI. In some embodiments, the target gene is edited. In some embodiments, plant with the target gene edited confer a trait selected from the group consisting of pest and pathogen resistance, insect resistance, plant-disease resistance, drought tolerance, flood tolerance, nutrient biofortification and high yield.

The present disclosure provides method for producing a gene-edited plant in which a mutation is introduced into a target gene on a genome and no exogenous gene is incorporated on the genome, the method comprising: (a) transforming the vector taught herein into a plant cell; (b) culturing the plant cell obtained in the step (a) and selecting a regenerated plant; and (c) selecting a plant in which at least one target gene is edited by a CRISPR/Cas system. In some embodiments, the vector comprises at least two gRNAs, each of which comprises at least 15 nucleotides guide sequence complementary to at least one genomic region of the target gene or functional derivative thereof. In some embodiments, said plant cell has one or more mutations in the genome which results in the reduced or abolished expression expression of the target gene as compared to said expression in a normal cell that does not have such mutations. In some embodiments, the target gene is edited and a plant with the target gene edited confers a trait selected from the group consisting of pest and pathogen resistance, insect resistance, plant-disease resistance, drought tolerance, flood tolerance, nutrient biofortification and high yield.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 demonstrates a diagram of a final product generated from ENABLE gene editing Kit, which is a gene-editing binary vector comprising of four expression cassettes: (i) HPTII gene driven by 35S promoter and its transcription is terminated by 35S terminator, (ii) SpCas9 gene driven by 2× 35S promoter and its transcription is terminated by NOS terminator, (iii) Target 1 guide RNA (gRNA) and gRNA backbone/scaffold driven by OsU6-2 promoter and its transcription is terminated by poly-T region, and (iv) Target 2 guide RNA (gRNA) and gRNA backbone/scaffold driven by OsU6-2 promoter and its transcription is terminated by poly-T region.

FIG. 2 demonstrates steps 1 of subcloning of two separate target gene gRNA sequences into gRNA Level 1 (L1) vectors in two individual tubes (tube 1 and tube 2). Two separate reactions take place in parallel in two separate tubes. Target 1 gRNA sequence is subcloned into the 1st gRNA expression cassette in tube 1 and Target 2 gRNA sequence is subcloned into the 2nd gRNA expression cassette in tube 2. Per gRNA there are two complementary primers, the gRNA-annealed primer pairs have 5′ overhangs to be swapped with lacZ gene by restriction and ligation reactions using BsaI and T4 DNA ligase in frame. Each expression cassette has unique fragment-specific sequence of the overhangs (e.g. generated by BbsI), which allows orderly assembly of multiple fragments.

FIG. 3 demonstrates step 2 of assembly of four expression cassettes into a destination L2 vector. In tube 3, four vectors (i.e. two gRNA L1 vectors from FIG. 2, one L1 vector for HPTII gene expression [pICSL11059], and one L1 vector for SpCas9 expression[pFH52]) are incubated with the L2 vector [pLCSL4723] as a backbone provider. Four expression cassettes from four L1 vectors are assembled into a L2 vector backbone using only the sequential or simultaneous activities of a single Type IIS restriction enzyme (i.e. BbsI) and T4 DNA ligase. BbsI cleaves DNA outside of the recognition sequence and creates unique overhangs. The multiple expression cassettes with overhangs allows their assembly in order into the vector back. The symbol * represent a unique overhang and the number refers to a complementary pair of the overhang for assembly by ligation. The destination L2 vector contains a red color selectable marker (CRed, containing an artificial bacterial operon responsible for canthaxanthin biosynthesis). This red color selectable marker comprises a cluster that contains the five crt genes, crtE, crtY, crtI, crtB, and crtW, necessary for the canthaxanthin biosynthesis. This selectable marker is designed to be replaced with the assembly of four expression cassettes.

FIG. 4 demonstrates a control vector comprising two gRNA targeting phytoene desaturase (PDS) in tube 4, which is generated from steps described in FIGS. 2-3.

FIGS. 5A-5B show vector maps of (1) pGMF1 L1 with an expression cassette comprising OsU6-2 promoter, lacZ gene, single guide RNA (sgRNA) scaffold, and Poly-T region (FIG. 5A) and (2) pGMF1 with an expression cassette comprising with OsU6-2 promoter, PDS target 1 gRNA sequence, single guide RNA (sgRNA) scaffold, and Poly-T region (FIG. 5B). In FIG. 5A, the lacZ gene can be replaced with gRNA sequence of interest. The lacZ gene is replaced with sgRNA1 targeting PDS in the expression cassette as shown in FIG. 5B. FIG. 5B is an illustration of one example of pGMF1 L1 vector with gRNA targeting PDS that is a control gene for the cloning kit of the present disclosure.

FIGS. 6A-6B show vector maps of (1) pGMF2 with an expression cassette comprising OsU6-2 promoter, lacZ gene, single guide RNA (sgRNA) scaffold, and Poly-T region (FIG. 6A) and (2) pGMF2 with an expression cassette comprising with OsU6-2 promoter, PDS target 2 gRNA sequence, single guide RNA (sgRNA) scaffold, and Poly-T region. In FIG. 6A, the lacZ gene can be replaced with gRNA sequence of interest. The lacZ gene is replaced with sgRNA2 targeting PDS in the expression cassette as shown in FIG. 6B.

FIG. 7 shows a vector map of pGMF3 L2 as a backbone provider, comprising pICSL4723 L2 backbone.

FIG. 8 shows a vector map of pGMF4 with an expression cassette comprising pFH52 2×CaMV 35S promoter, Arabidopsis optimized SpCas9 gene, and NOS terminator.

FIG. 9 shows a vector map of pGMF5 with an expression cassette comprising pICSL11059 CaMV 35S promoter, hygromycin phosphotransferase II (HPTII) gene and 35S terminator.

FIG. 10 shows a binary vector map of Final L2 for targeting PDS comprising four expression cassettes (i) from pGMF1 with PDS target 1 sgRNA (FIG. 5B), (ii) from pGMF2 with PDS target 2 sgRNA (FIG. 6B), (iii) from pGMF4 (FIG. 8) with Arabidopsis optimized SpCas9 gene, and (iv) from pGMF5 with HPTII gene (FIG. 9). The Final L2 vector backbone is from pGMF3.

FIG. 11 shows a vector map of pGMF6 with an expression cassette comprising CaMV 35S promoter, eGFP gene, N7-NLS and 35S terminator.

FIG. 12 shows a binary vector map of Final L2 for targeting PDS comprising four expression cassettes (i) from pGMF1 with PDS target 1 sgRNA (FIG. 5B), (ii) from pGMF2 with PDS target 2 sgRNA (FIG. 6B), (iii) from pGMF4 (FIG. 8), and (iv) from pGMF6 (FIG. 11). The Final L2 vector backbone is from pGMF3.

FIG. 13 shows a sequence information (SEQ ID NO: 14) of an expression cassette of the pGMF1 L1 subcloning vector for gRNA1. The left border (LB) and right border (RB) sequences are shaded with light grey. BbsI enzyme restriction site (GAAGAC) is in bold. BsaI enzyme restriction site (GAGACC) is italicized in bold. The lacZ gene sequence to be swapped with gRNA of interest is shaded with dark grey. gRNA scaffold sequence (SEQ ID NO: 2) is also shaded and italicized in bold. The transcription stop sequence (TTTTTTTTT) is in bold.

FIG. 14 shows a sequence information (SEQ ID NO: 15) of an expression cassette of the pGMF1 L1 subcloning vector for gRNA2. The left border (LB) and right border (RB) sequences are shaded with light grey. BbsI enzyme restriction site (GAAGAC) is in bold. BsaI enzyme restriction site (GAGACC) is italicized in bold. The lacZ gene sequence to be swapped with gRNA of interest is shaded with dark grey. gRNA scaffold sequence (SEQ ID NO: 2) is also shaded and italicized in bold. The transcription stop sequence (TTTTTTTTT) is in bold.

FIG. 15A shows a example of DNA sequence of target gene (sense strand; SEQ ID NO:3 and antisense strand; SEQ ID NO: 4) with two protospacers targeted by guide sequences of two gRNAs for CRISPR/Cas9 insertions and deletions (indels). FIG. 15B shows 5′ gRNA1 with PAM (SEQ ID NO: 5) and two primer sequences to make gRNA1 annealed primer pairs with overhangs with overhangs at 5′ (SEQ ID NOs: 6 and 7). FIG. 15C shows 3′ gRNA2 with PAM (SEQ ID NO: 8) and 5′ reverse-complemented gRNA2 with PAM (SEQ ID NO: 9) and two primer sequences to make gRNA 2 annealed primer pairs with overhangs at each 5′ (SEQ ID NOs: 10 and 11).

FIG. 16 illustrates pre-steps for designing two gRNAs, each of which comprises a guide sequence binding to a target DNA.

FIGS. 17A-17J illustrate diagrams of gene-editing cloning steps; (1) recovering plasmids (including pGMF1, pGMF2, pGMF3, pGMF4, pGMF5, and pGMF6 (FIG. 17A); (2) selecting plasmids with inserts of interest for cloning (FIG. 17B); (3) preparing two gRNAs comprising the guide sequence of interest, respectively (FIGS. 17C and 17D); (4) cloning binary vector (FIGS. 17E and 17F); (5) verifying a final vector plasmid (FIG. 17G); (6) checking transient transformation (FIG. 17H); (7) producing stable transformation for trait (FIG. 17I); (8) confirming desired gene edit(s) in plants (FIG. 17J).

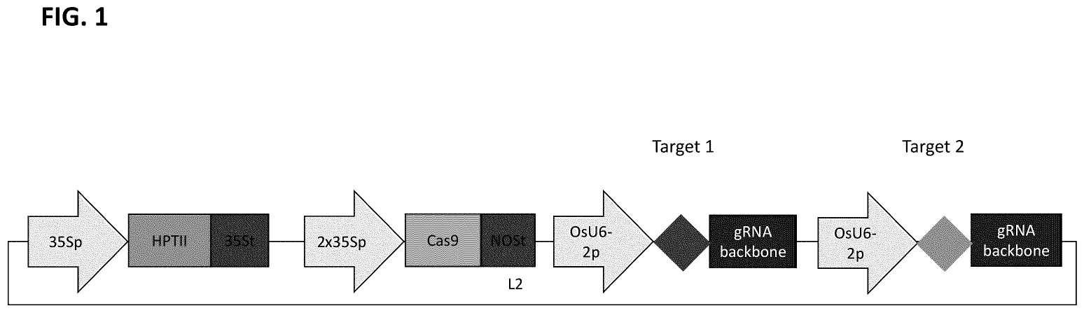

### DETAILED DESCRIPTION

The present disclosure provides a gene editing cloning system and/or kit using CRISPR-Cas for targeted knockout in plants of interest. The gene editing cloning system and/or kit is used for the plants to acquire desired traits, such as insect and disease resistance, nutrient biofortification and drought and flood tolerance. The present disclosure provides gene edited plants, plant parts, and plant cells wherein native genes have been altered to obtain beneficial traits, and methods of making the same. The present disclosure provides use of the gene editing cloning systems and/or kit for the generation of plants that possess desired trait(s) when compared to wildtype type plants without any gene(s) of target edited.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Following long-standing patent law convention, the terms “a,” “an,” and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Throughout this specification, unless the context requires otherwise, the words “comprise”, or variations such as “comprises” or “comprising”, will be understood to imply the inclusion of a stated element or integer or group of elements or integers but not the exclusion of any other element or integer or group of elements or integers.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of ±10% from the specified amount, as such variations are appropriate to perform the disclosed methods and/or employ the disclosed compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, BCD and ABCD). In some embodiments, one or more of the elements to which the “and/or” refers can also individually be present in single or multiple occurrences in the combinations(s) and/or subcombination(s).

As used herein, the term “plant” can refer to any living organism belonging to the kingdom Plantae (i.e., any genus/species in the Plant Kingdom), to a whole plant, any part thereof, or a cell or tissue culture derived from a plant. Thus, the term “plant” can refer to any of whole plants, plant components or organs (e.g., leaves, stems, roots, etc.), plant tissues, seeds and/or plant cells.

As used herein, the term “plant cell” is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores.

The term “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “allele” is used both as it is known in the art as one of two or more versions of a gene or peptide, and also to refer to synthetic variants of a gene or peptide containing one or more changes from the native sequence.

As used herein, the term “codon optimization” implies that the codon usage of a DNA or RNA is adapted to that of a cell or organism of interest to improve the transcription rate of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism.

As used herein, the term “endogenous” or “endogenous gene,” refers to the naturally occurring gene, in the location in which it is naturally found within the host cell genome. “Endogenous gene” is synonymous with “native gene” as used herein. An endogenous gene as described herein can include alleles of naturally occurring genes that have been mutated according to any of the methods of the present disclosure, i.e. an endogenous gene could have been modified at some point by traditional plant breeding methods and/or next generation plant breeding methods.

As used herein, the term “exogenous” refers to a substance coming from some source other than its native source. For example, the terms “exogenous protein,” or “exogenous gene” refer to a protein or gene from a non-native source, and that has been artificially supplied to a biological system. As used herein, the term “exogenous” is used interchangeably with the term “heterologous,” and refers to a substance coming from some source other than its native source.

As used herein, the term “heterologous” refers to a substance coming from some source or location other than its native source or location. In some embodiments, the term “heterologous nucleic acid” refers to a nucleic acid sequence that is not naturally found in the particular organism. For example, the term “heterologous promoter” may refer to a promoter that has been taken from one source organism and utilized in another organism, in which the promoter is not naturally found. However, the term “heterologous promoter” may also refer to a promoter that is from within the same source organism, but has merely been moved to a novel location, in which said promoter is not normally located.

Heterologous gene sequences can be introduced into a target cell by using an “expression vector,” which can be a plant expression vector, for example a plant expression vector. Methods used to construct vectors are well known to a person skilled in the art and described in various publications. In particular, techniques for constructing suitable vectors, including a description of the functional components such as promoters, enhancers, termination and polyadenylation signals, selection markers, origins of replication, and splicing signals, are reviewed in the prior art. Vectors may include but are not limited to plasmid vectors, phagemids, cosmids, artificial/mini-chromosomes (e.g. ACE), or viral vectors such as baculovirus, retrovirus, adenovirus, adeno-associated virus, herpes simplex virus, retroviruses, bacteriophages. The plant expression vectors will typically contain also prokaryotic sequences that facilitate the propagation of the vector in bacteria such as an origin of replication and antibiotic resistance genes for selection in bacteria. A variety of plant expression vectors, containing a cloning site into which a polynucleotide can be operatively linked, are well known in the art and some are commercially available from companies such as Stratagene, La Jolla, Calif.; Invitrogen, Carlsbad, Calif.; Promega, Madison, Wis. or BD Biosciences Clontech, Palo Alto, Calif. In one embodiment the expression vector comprises at least one nucleic acid sequence which is a regulatory sequence necessary for transcription and translation of nucleotide sequences that encode for a peptide/polypeptide/protein of interest.

As used herein, the term “homologous” or “homolog” is used as it is known in the art and refers to related sequences that share a common ancestor. The homolog are thought or known to be functionally related. A functional relationship may be indicated in any one of a number of ways, including, but not limited to: (a) degree of sequence identity and/or (b) the same or similar biological function. Preferably, both (a) and (b) are indicated. The degree of sequence identity may vary, but in one embodiment, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. The term “homolog” is sometimes used to apply to the relationship between genes separated by the event of speciation (“ortholog”) or to the relationship between genes separated by the event of genetic duplication within the same species (“paralog”). Homology can be determined using software programs readily available in the art, such as those discussed in Current Protocols in Molecular Biology (F. M. Ausubel et al., eds., 1987) Supplement 30, section 7.718, Table 7.71.

The term “homeolog” refers to a homeologous gene or chromosome, resulting from polyploidy or chromosomal duplication events. This contrasts with the more common ‘homolog’, which is defined immediately above.

The terms “genetically engineered host cell,” “recombinant host cell,” and “recombinant strain” are used interchangeably herein and refer to host cells that have been genetically engineered by the methods of the present disclosure. Thus, the terms include a host cell (e.g., bacteria, yeast cell, fungal cell, CHO, human cell, plant cell, protoplast derived from plant, callus, etc.) that has been genetically altered, modified, or engineered, such that it exhibits an altered, modified, or different genotype and/or phenotype (e.g., when the genetic modification affects coding nucleic acid sequences), as compared to the naturally-occurring host cell from which it was derived. It is understood that the terms refer not only to the particular recombinant host cell in question, but also to the progeny or potential progeny of such a host cell.

As used herein, the term “naturally occurring” as applied to a nucleic acid, a polypeptide, a cell, or an organism, refers to a nucleic acid, polypeptide, cell, or organism that is found in nature. The term “naturally occurring” may refer to a gene or sequence derived from a naturally occurring source. Thus, for the purposes of this disclosure, a “non-naturally occurring” sequence is a sequence that has been synthesized, mutated, engineered, edited, or otherwise modified to have a different sequence from known natural sequences. In some embodiments, the modification may be at the protein level (e.g., amino acid substitutions). In other embodiments, the modification may be at the DNA level (e.g., nucleotide substitutions).

The term “next generation plant breeding” refers to a host of plant breeding tools and methodologies that are available to today's breeder. A key distinguishing feature of next generation plant breeding is that the breeder is no longer confined to relying upon observed phenotypic variation, in order to infer underlying genetic causes for a given trait. Rather, next generation plant breeding may include the utilization of molecular markers and marker assisted selection (MAS), such that the breeder can directly observe movement of alleles and genetic elements of interest from one plant in the breeding population to another, and is not confined to merely observing phenotype. Further, next generation plant breeding methods are not confined to utilizing natural genetic variation found within a plant population. Rather, the breeder utilizing next generation plant breeding methodology can access a host of modern genetic engineering tools that directly alter/change/edit the plant's underlying genetic architecture in a targeted manner, in order to bring about a phenotypic trait of interest. In aspects, the plants bred with a next generation plant breeding methodology are indistinguishable from a plant that was bred in a traditional manner, as the resulting end product plant could theoretically be developed by either method. In particular aspects, a next generation plant breeding methodology may result in a plant that comprises: a genetic modification that is a deletion or insertion of any size; a genetic modification that is one or more base pair substitution; a genetic modification that is an introduction of nucleic acid sequences from within the plant's natural gene pool (e.g. any plant that could be crossed or bred with a plant of interest) or from editing of nucleic acid sequences in a plant to correspond to a sequence known to occur in the plant's natural gene pool; and offspring of said plants.

The terms “polynucleotide,” “nucleic acid,” and “nucleotide sequence,” used interchangeably herein, refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. This term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. It also includes modified nucleic acids such as methylated and/or capped nucleic acids, nucleic acids containing modified bases, backbone modifications, and the like. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” “nucleic acid,” and “nucleotide sequence” should be understood to include, as applicable to the embodiments being described, single-stranded (such as sense or antisense) and double-stranded polynucleotides.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “sequence identity” or “sequence homology” or “sequence similarity” refers to the presence of identical nucleotides or amino acids at corresponding positions of two sequences. Readily available sequence comparison and multiple sequence alignment algorithms are, respectively, the Basic Local Alignment Search Tool (BLAST®) and ClustalW/ClustalW2/Clustal Omega programs available on the Internet (e.g., the website of the EMBL-EBI). Some alignment programs are MacVector (Oxford Molecular Ltd, Oxford, U.K.) and ALIGN Plus (Scientific and Educational Software, Pennsylvania). Other non-limiting alignment programs include Sequencher (Gene Codes, Ann Arbor, Mich.), AlignX, and Vector NTI (Invitrogen, Carlsbad, Calif.). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001. An example of a local alignment algorithm utilized for the comparison of sequences is the NCBI Basic Local Alignment Search Tool (BLAST®) (Altschul et al. 1990 J. Mol. Biol. 215: 403-10), which is available from several sources, including the National Center for Biotechnology Information (NCBI, Bethesda, Md.) and on the Internet, for use in connection with the sequence analysis programs blastp, blastn, blastx, tblastn and tblastx. It can be accessed on the internet via the National Library of Medicine (NLM)'s world-wide-web URL. A description of how to determine sequence identity using this program is available at the NLM's website on BLAST tutorial. Another example of a mathematical algorithm utilized for the global comparison of sequences is the Clustal W and Clustal X (Larkin et al. 2007 Bioinformatics, 23, 2947-294, Clustal W and Clustal X version 2.0) as well as Clustal omega. Unless otherwise stated, references to sequence identity used herein refer to the NCBI Basic Local Alignment Search Tool (BLAST®).

As used herein, the phrases “DNA construct”, “chimeric construct”, “construct”, and “recombinant DNA construct” are used interchangeably herein. A recombinant DNA construct comprises an artificial combination of nucleic acid fragments, e.g., regulatory and coding sequences that are not found together in nature. For example, a construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA, a guide RNA or a protein (precursor or mature).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example a guide RNA (gRNA), a CRISPR RNA (crRNA), a trans-acting CRISPR RNA (tracrRNA), a single guide RNA (sgRNA), an antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development in animal and/or plant.

In some embodiments, a final vector of the present disclosure comprises at least one expression cassette comprising DNA sequence capable of directing expression of (1) at least one guide RNA comprised of one guide sequence of interest with a gRNA scaffold sequence.

In some embodiments, a final vector of the present disclosure comprises at least two expression cassettes, (1) the first cassette comprising DNA sequence capable of directing expression of (1) a first guide RNA comprised of one guide sequence of interest with a gRNA scaffold sequence; and (2) the second cassette comprising DNA sequence capable of directing expression of a second one guide RNA comprised of one guide sequence of interest with a gRNA scaffold sequence.

In some embodiments, a final vector of the present disclosure comprises at least three expression cassettes; (1) the first cassette comprising DNA sequence capable of directing expression of a first guide RNA comprised of one guide sequence of interest with a gRNA scaffold sequence; (2) the second cassette comprising DNA sequence capable of directing expression of a second one guide RNA comprised of one guide sequence of interest with a gRNA scaffold sequence; and (3) a gene encoding CRISPR-associated (Cas) protein.

In some embodiments, a final vector of the present disclosure comprises at least four expression cassettes; (1) the first cassette comprising DNA sequence capable of directing expression of a first guide RNA comprised of one guide sequence of interest with a gRNA scaffold sequence; (2) the second cassette comprising DNA sequence capable of directing expression of a second one guide RNA comprised of one guide sequence of interest with a gRNA scaffold sequence; (3) a gene encoding CRISPR-associated (Cas) protein; and (4) at least one selectable marker which is Ampicillin antibiotic resistance gene, Kanamycin antibiotic resistance gene, or hygromycin resistance gene.

In other embodiments, a final vector of the present disclosure comprises at least five expression cassettes; (1) the first cassette comprising DNA sequence capable of directing expression of a first guide RNA comprised of one guide sequence of interest with a gRNA scaffold sequence; (2) the second cassette comprising DNA sequence capable of directing expression of a second one guide RNA comprised of one guide sequence of interest with a gRNA scaffold sequence; (3) a gene encoding CRISPR-associated (Cas) protein; and (4) at least two selectable markers including (i) Ampicillin antibiotic resistance gene, Kanamycin antibiotic resistance gene, or hygromycin resistance gene and (ii) GFP or eGFP.

In further embodiments, a final vector of the present disclosure comprises multiple expression cassettes, which include combinations of DNA sequences capable of directing expression of (1) the first cassette comprising DNA sequence capable of directing expression of a first guide RNA comprised of one guide sequence of interest with a gRNA scaffold sequence; (2) the second cassette comprising DNA sequence capable of directing expression of a second one guide RNA comprised of one guide sequence of interest with a gRNA scaffold sequence; (3) a gene encoding CRISPR-associated (Cas) protein; and (4) at least two selectable markers including (i) Ampicillin antibiotic resistance gene, Kanamycin antibiotic resistance gene, or hygromycin resistance gene and (ii) GFP or eGFP.

In some embodiments, a first expression cassette comprises DNA sequence capable of directing expression of (i) at least one guide RNA comprised of one guide sequence of interest with a gRNA scaffold sequence. In some embodiments, a second expression cassette comprises DNA sequence capable of directing expression of (ii) at least one guide RNA comprised of one guide sequence of interest with a gRNA scaffold sequence. In some embodiments, a second expression cassette comprises DNA sequence capable of directing expression of (iii) a gene encoding CRISPR-associated (Cas) protein. In some embodiments, a second expression cassette comprises DNA sequence capable of directing expression of (iv) at least one selectable marker selected from Ampicillin antibiotic resistance gene, Kanamycin antibiotic resistance gene, and hygromycin resistance gene In some embodiments, a second expression cassette comprises DNA sequence capable of directing expression of (v) at least one selectable marker including GFP and eGFP.

As used herein “cisgene” refers to a gene from the same species, or a species closely related enough to be conventionally bred. “Transgene” refers to a gene from a different species, and may also be referred to as “heterologous” (an amino acid or a nucleic acid sequence which is not naturally found in the particular organism). Both transgenes and heterologous sequences would be considered “exogenous” as referring to a substance coming from some source other than its native source.

The term “operably linked” refers to the juxtaposition of two or more components (such as sequence elements) having a functional relationship. For example, the sequential arrangement of the promoter polynucleotide with a further oligo- or polynucleotide, resulting in transcription of the further polynucleotide.

As used herein, “promoter” refers to a DNA sequence capable of controlling the expression of a coding sequence or functional RNA. In some embodiments, the promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter.

As used herein, “selectable marker” is a nucleic acid segment that allows one to select for a molecule (e.g., a plasmid) or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

As used herein, the term “nucleotide change” or “nucleotide modification” refers to, e.g., nucleotide substitution, deletion, and/or insertion, as is well understood in the art. For example, such nucleotide changes/modifications include mutations containing alterations that produce silent substitutions, additions, or deletions, but do not alter the properties or activities of the encoded protein or how the proteins are made. As another example, such nucleotide changes/modifications include mutations containing alterations that produce replacement substitutions, additions, or deletions, that alter the properties or activities of the encoded protein or how the proteins are made.

As used herein, the term “protein modification” refers to, e.g., amino acid substitution, amino acid modification, deletion, and/or insertion, as is well understood in the art.

A “CRISPR-associated effector” or “CRISPR-associated protein” or “CRISPR enzyme” as used herein can thus be defined as any nuclease, nickase, or recombinase associated with the CRISPR (Clustered Regularly Interspaced Short Palindromic Repeats), having the capacity to introduce a single- or double-strand cleavage into a genomic target site, or having the capacity to introduce a targeted modification, including a point mutation, an insertion, or a deletion, into a genomic target site of interest. At least one CRISPR-associated effector can act on its own, or in combination with other molecules as part of a molecular complex. The CRISPR-associated effector can be present as fusion molecule, or as individual molecules associating by or being associated by at least one of a covalent or non-covalent interaction with gRNA and/or target site so that the components of the CRISPR-associated complex are brought into close physical proximity.

The term “Cas9 nuclease” and “Cas9” can be used interchangeably herein, which refer to a RNA-guided DNA endonuclease enzyme associated with the CRISPR (Clustered Regularly Interspaced Short Palindromic Repeats), including the Cas9 protein or fragments thereof (such as a protein comprising an active DNA cleavage domain of Cas9 and/or a gRNA binding domain of Cas9). Cas9 is a component of the CRISPR/Cas genome editing system, which targets and cleaves a DNA target sequence to form a DNA double strand breaks (DSB) under the guidance of a guide RNA. Cas9 is one example of the CRISPR-associated effectors/proteins.

The term “CRISPR RNA” or “crRNA” refers to the RNA strand responsible for hybridizing with target DNA sequences, and recruiting CRISPR endonucleases and/or CRISPR-associated effectors. crRNAs may be naturally occurring, or may be synthesized according to any known method of producing RNA.

The term “tracrRNA” refers to a small trans-encoded RNA. TracrRNA is complementary to and base pairs with crRNA to form a crRNA/tracrRNA hybrid, capable of recruiting CRISPR endonucleases and/or CRISPR-associated effectors to target sequences. tracrRNAs may be naturally occurring, or may be synthesized according to any known method of producing RNA.

The term “Guide RNA” or “gRNA” as used herein refers to an RNA sequence or combination of sequences that recognized a target DNA region of interest and direct a CRISPR endonuclease and/or CRISPR-associated effectors to the target DNA sequence for editing. The gRNA is made up of two parts: crispr RNA (crRNA), about 17-20 nucleotide sequence complementary to the target DNA, and a tracr RNA, which serves as a binding scaffold for the Cas nuclease. Typically gRNA is composed of crRNA and tracrRNA molecules forming complexes through partial complement, wherein crRNA comprises a sequence that is sufficiently complementary to a target sequence for hybridization and directs the CRISPR complex (i.e. Cas9-crRNA/tracrRNA hybrid) to specifically bind to the target sequence. Also, single guide RNA (sgRNA) can be designed, which comprises the characteristics of both crRNA and tracrRNA. Therefore, as used herein, a guide RNA can be a natural or synthetic crRNA (e.g., for Cpf1), a natural or synthetic crRNA/tracrRNA hybrid (e.g., for Cas9), or a single-guide RNA (sgRNA).

The term “guide RNA sequence” or “guide sequence” refers to the portion of a crRNA or guide RNA (gRNA) that is responsible for hybridizing with the target DNA. In some embodiments, gRNA refers to about 17-23 nucleotide sequence complementary to the target DNA, which corresponds to crRNA part. In other embodiments, “gRNA scaffold sequence” or “gRNA scaffold” or “gRNA backbone” refers to a nucleotide sequence (e.g. SEQ ID NO: 2) that serves a binding scaffold for the Cas nuclease.

The term “protospacer” refers to the DNA sequence targeted by a guide sequence of crRNA or gRNA. In some embodiments, the protospacer sequence hybridizes with the crRNA or gRNA guide (spacer) sequence of a CRISPR complex.

The term “CRISPR landing site” as used herein, refers to a DNA sequence capable of being targeted by a CRISPR-Cas complex. In some embodiments, a CRISPR landing site comprises a proximately placed protospacer/Protopacer Adjacent Motif combination sequence that is capable of being cleaved by a CRISPR complex.

The term “CRISPR complex”, “CRISPR endonuclease complex”, “CRISPR Cas complex”, or “CRISPR-gRNA complex” are used interchangeably herein. “CRISPR complex” refers to a Cas9 nuclease and/or a CRISPR-associated effectors complexed with a guide RNA (gRNA). The term “CRISPR complex” thus refers to a combination of CRISPR endonuclease and guide RNA capable of inducing a double stranded break at a CRISPR landing site. In some embodiments, “CRISPR complex” of the present disclosure refers to a combination of catalytically dead Cas9 protein and guide RNA capable of targeting a target sequence, but not capable of inducing a double stranded break at a CRISPR landing site because it loses a nuclease activity. In other embodiments, “CRISPR complex” of the present disclosure refers to a combination of Cas9 nickase and guide RNA capable of introducing gRNA-targeted single-strand breaks in DNA instead of the double-strand breaks created by wild type Cas enzymes.

As used herein, the term “directing sequence-specific binding” in the context of CRISPR complexes refers to a guide RNA's ability to recruit a CRISPR endonuclease and/or a CRISPR-associated effectors to a CRISPR landing site.

As used herein the term “targeted” refers to the expectation that one item or molecule will interact with another item or molecule with a degree of specificity, so as to exclude non-targeted items or molecules. For example, a first polynucleotide that is targeted to a second polynucleotide, according to the present disclosure has been designed to hybridize with the second polynucleotide in a sequence specific manner (e.g., via Watson-Crick base pairing). In some embodiments, the selected region of hybridization is designed so as to render the hybridization unique to the one, or more targeted regions. A second polynucleotide can cease to be a target of a first targeting polynucleotide, if its targeting sequence (region of hybridization) is mutated, or is otherwise removed/separated from the second polynucleotide. Furthermore, “targeted” can be interchangeably used with “site-specific” or “site-directed,” which refers to an action of molecular biology which uses information on the sequence of a genomic region of interest to be modified, and which further relies on information of the mechanism of action of molecular tools, e.g., nucleases, including CRISPR nucleases and variants thereof, TALENs, ZFNs, meganucleases or recombinases, DNA-modifying enzymes, including base modifying enzymes like cytidine deaminase enzymes, histone modifying enzymes and the like, DNA-binding proteins, cr/tracr RNAs, guide RNAs and the like.

The term “seed region” refers to the critical portion of a crRNA's or guide RNA's guide sequence that is most susceptible to mismatches with their targets. In some embodiments, a single mismatch in the seed region of a crRNA/gRNA can render a CRISPR complex inactive at that binding site. In some embodiments, the seed regions for Cas9 endonucleases are located along the last ˜12 nts of the 3′ portion of the guide sequence, which correspond (hybridize) to the portion of the protospacer target sequence that is adjacent to the PAM. In some embodiments, the seed regions for Cpf1 endonucleases are located along the first ˜5 nts of the 5′ portion of the guide sequence, which correspond (hybridize) to the portion of the protospacer target sequence adjacent to the PAM.

“Complementary” refers to the capacity for pairing, through base stacking and specific hydrogen bonding, between two sequences comprising naturally or non-naturally occurring bases or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and Santa Lucia, Nucl. Acids Research, 2005; 33 (19): 6258-6267.

As referred to herein, a “complementary nucleic acid sequence” is a nucleic acid sequence comprising a sequence of nucleotides that enables it to non-covalently bind to another nucleic acid in a sequence-specific, antiparallel, manner (i.e., a nucleic acid specifically binds to a complementary nucleic acid) under the appropriate in vitro and/or in vivo conditions of temperature and solution ionic strength.

The term “modified” refers to a substance or compound (e.g., a cell, a polynucleotide sequence, and/or a polypeptide sequence) that has been altered or changed as compared to the corresponding unmodified substance or compound.

“Isolated” refers to a material that is free to varying degrees from components which normally accompany it as found in its native state.

The term “gene edited plant, part or cell” as used herein refers to a plant, part or cell that comprises one or more endogenous genes that are edited by a gene editing system. The gene editing system of the present disclosure comprises a targeting element and/or an editing element. The targeting element is capable of recognizing a target genomic sequence. The editing element is capable of modifying the target genomic sequence, e.g., by substitution or insertion of one or more nucleotides in the genomic sequence, deletion of one or more nucleotides in the genomic sequence, alteration of genomic sequences to include regulatory sequences, insertion of transgenes at a safe harbor genomic site or other specific location in the genome, or any combination thereof. The targeting element and the editing element can be on the same nucleic acid molecule or different nucleic acid molecules. In some embodiments, the gene edited plant, part or cell has the downregulated, suppressed, reduced, or abolished expression of the target gene that is edited by the gene editing system of the present disclosure.

As used herein, “guide RNA annealed primer pairs” or “gRNA annealed primer pairs” refers to two oligo/primers with the gRNA sequence (if 5′ from the PAM site) or reverse complemented gRNA sequence (if 3′) with four base pair overhangs. GTTG added to the 5′ and AAAC to the 3′ complementary gRNA sequence. These overhangs and sequence orientations allow for directional subcloning of the gRNA.

### Overview

CRISPR/Cas is a commonly used tool for editing genomes in various organisms, including plants. However, it often requires multiple genetic elements to be delivered into plants, which requires a quick and easy cloning process. Modular cloning based on the Golden Gate method has allowed for the development of cloning systems with standardized genetic parts (such as promoters, coding sequences, and terminators) that can be easily exchanged and assembled into expression units. These units can then be further assembled into more complex, multigene constructs.

While generating gene knockouts has become easy, more sophisticated applications, such as allele replacements or targeted gene insertions, remain challenging due to low efficiency of homology-directed repair (HDR) in plants. Typically, co-expressing the CRISPR/Cas nuclease and its cognate guide RNA is required for targeted mutagenesis in plants. The CRISPR/Cas system allows for multiplexing, meaning that DNA can be targeted at multiple genomic locations by co-expressing multiple guide RNAs specific to those loci.

As genome editing applications in plants often require delivering multiple expression units into plant cells, including a selectable marker, a CRISPR/Cas nuclease-encoding gene, and one or more guide RNAs, there is a need for a quick and easy way to assemble DNA constructs encoding such expression units.

The present disclosure provides an efficient and easy hand-on cloning protocol, system, and kits for CRISPR/Cas9 directed insertions and deletions (indels) in planta, thereby conferring desired trait(s) to the plants of interest. The present disclosure provides necessary components for the gene editing cloning system for plants including orphan crops. The components include, but are not limited to (i) at least two guide RNAs for targeted mutagenesis in monocot or dicot, (ii) plant codon optimized Cas9 endonuclease, and/or (iii) selectable markers for plant selection.

The present disclosure teaches the gene-editing cloning system based on the Golden Gate cloning method, which provides a means for quick and facile assembly of multi-expression unit constructs using standard genetic parts. Inventors have modified and expanded Golden Gate cloning system to develop the gene editing system and/or kit of the present disclosure that enables to assemble the final constructs in a quick, easy, and efficient manner. This system and/or kit can be utilized for genome editing applications in plants in monocot and dicot plants.

The present disclosure teaches the gene editing kit of the present disclosure, which includes (1) vectors, for examples, pGMF1 Subcloning vector for gRNA1 Pol III promoter; pGMF2 subcloning vector for gRNA2 Pol III promoter; pGMF3 vector with Golden Gate Level 2 Backbone; pGMF4 vector with codon-optimized SpCas9 gene; pGMF5 vector with hygromycin resistance marker for transformed plant selection; pGMF6 vector with nuclear localized eGFP marker for monocot and dicot; (2) Premixed Buffer 1: T4 DNA Ligase Buffer; (3) Bovine Serum Albumin; (4) Double-distilled nuclease-free water; (5) Enzyme Mix A: Type II RE BsaI-HFv2 (20 U/μl) and T4 DNA Ligase (400,000 U/μl; (6) Enzyme Mix B: Type II RE BbsI-HF (20 U/μl) and T4 DNA Ligase (400,000 U/μl); and (7) Control gRNA1 annealed primer pairs.

The present disclosure teaches that a two step cloning process that combines all vectors, allows for the subcloning of two guide RNA into one complete binary vector for agrobacterium-mediated or biolistic bombardment transformation in plants. To ensure the simplest cloning procedure of two guide RNAs, and the ability to assess transformation efficiency, three new vectors have been developed and provided herewith, two of which are for guide RNA positional subcloning (pGMF1 and pGMF2), and another for eGFP nucleo-localization (pGMF6) in planta for evaluating transformation efficiency.

The present disclosure provides gene editing cloning kits available to perform in planta CRISPR/Cas9 with at least two guide RNAs, and the reagents necessary for molecular cloning.

### Gene Editing

As used herein, the term “gene editing system” refers to a system comprising one or more DNA-binding domains or components and one or more DNA-modifying domains or components, or isolated nucleic acids, e.g., one or more vectors, encoding said DNA-binding and DNA-modifying domains or components. Gene editing systems are used for modifying the nucleic acid of a target gene and/or for modulating the expression of a target gene. In known gene editing systems, for example, the one or more DNA-binding domains or components are associated with the one or more DNA-modifying domains or components, such that the one or more DNA-binding domains target the one or more DNA-modifying domains or components to a specific nucleic acid site. Methods and compositions for enhancing gene editing is well known in the art. See example, U.S. Patent Application Publication No. 2018/0245065, which is incorporated by reference in its entirety.

Certain gene editing systems are known in the art, and include but are not limited to, zinc finger nucleases, transcription activator-like effector nucleases (TALENs); clustered regularly interspaced short palindromic repeats (CRISPR)/Cas systems, meganuclease systems, and viral vector-mediated gene editing.

In some embodiments, the present disclosure teaches methods for gene editing/cloning utilizing DNA nucleases. CRISPR complexes, transcription activator-like effector nucleases (TALENs), zinc finger nucleases (ZFNs), and FokI restriction enzymes, which are some of the sequence-specific nucleases that have been used as gene editing tools. These enzymes are able to target their nuclease activities to desired target loci through interactions with guide regions engineered to recognize sequences of interest. In some embodiments, the present disclosure teaches CRISPR-based gene editing methods to genetically engineer the genome of plant species of the present disclosure in order to stimulate, enhance, or modulate suberin content of plant cells, plant tissues, plant parts or whole plants.

CRISPR-Cas system(s) (e.g., single or multiplexed) can be used in conjunction with recent advances in crop genomics. Such CRISPR-Cas system(s) can be used to perform efficient and cost effective plant gene or genome interrogation or editing or manipulation—for instance, for rapid investigation and/or selection and/or interrogations and/or comparison and/or manipulations and/or transformation of plant genes or genomes; e.g., to create, identify, develop, optimize, or confer trait(s) or characteristic(s) to plant(s) or to transform a plant genome. There can accordingly be improved production of plants, new plants with new combinations of traits or characteristics or new plants with enhanced traits. Such CRISPR-Cas system(s) can be used with regard to plants in Site-Directed Integration or Gene Editing or any Near Reverse Breeding or Reverse Breeding techniques. Embodiments of the disclosure can be used in genome editing in plants or where RNAi or similar genome editing techniques have been used previously; see, e.g., Nekrasov, “Plant genome editing made easy: targeted mutagenesis in model and crop plants using the CRISPR/Cas system,” Plant Methods 2013, 9:39 (doi:10.1186/1746-4811-9-39); Brooks, “Efficient gene editing in tomato in the first generation using the CRISPR/Cas9 system,” Plant Physiology September 2014 pp 114.247577; Shan, “Targeted genome modification of crop plants using a CRISPR-Cas system,” Nature Biotechnology 31, 686-688 (2013); Feng, “Efficient genome editing in plants using a CRISPR/Cas system,” Cell Research (2013) 23:1229-1232. doi:10.1038/cr.2013.114; published online 20 Aug. 2013; Xie, “RNA-guided genome editing in plants using a CRISPR-Cas system,” Mol Plant. 2013 November; 6(6):1975-83. doi: 10.1093/mp/sst119. Epub 2013 Aug. 17; Xu, “Gene targeting using the Agrobacterium tumefaciens-mediated CRISPR-Cas system in rice,” Rice 2014, 7:5 (2014), Zhou et al., “Exploiting SNPs for biallelic CRISPR mutations in the outcrossing woody perennial Populus reveals 4-coumarate: CoA ligase specificity and Redundancy,” New Phytologist (2015) (Forum) 1-4 (available online only at newphytologist.com); Caliando et al, “Targeted DNA degradation using a CRISPR device stably carried in the host genome, NATURE COMMUNICATIONS 6:6989, U.S. Pat. No. 6,603,061—Agrobacterium-Mediated Plant Transformation Method; U.S. Pat. No. 7,868,149—Plant Genome Sequences and Uses Thereof and US 2009/0100536—Transgenic Plants with Enhanced Agronomic Traits, all the contents and disclosure of each of which are herein incorporated by reference in their entirety. In the practice of the disclosure, the contents and disclosure of Morrell et al “Crop genomics: advances and applications,” Nat Rev Genet. 2011 Dec. 29; 13(2):85-96; each of which is incorporated by reference herein including as to how herein embodiments may be used as to plants.

(i) CRISPR Systems

CRISPR (Clustered Regularly Interspaced Short Palindromic Repeats) and CRISPR-associated (cas) endonucleases were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers). (Wiedenheft, B., et. al. Nature. 2012; 482:331; Bhaya, D., et. al., Annu. Rev. Genet. 2011; 45:231; and Terms, M. P. et. al., Curr. Opin. Microbiol. 2011; 14:321). Bacteria and archaea possessing one or more CRISPR loci respond to viral or plasmid challenge by integrating short fragments of foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz, R. E., et. al., Science. 2012:329; 1355; Gesner, E. M., et. al., Nat. Struct. Mol. Biol. 2001, 18:688; Jinek, M., et. al., Science. 2012:337; 816-21). Target recognition by crRNAs occurs through complementary base pairing with target DNA, which directs cleavage of foreign sequences by means of Cas proteins. (Jinek et. al. 2012 “A Programmable dual-RNA-guided DNA endonuclease in adaptive bacterial immunity.” Science. 2012:337; 816-821).

There are at least five main CRISPR system types (Type I, II, III, IV and V) and at least 16 distinct subtypes (Makarova, K. S., et al., Nat Rev Microbiol. 2015. Nat. Rev. Microbiol. 13, 722-736). CRISPR systems are also classified based on their effector proteins. Class 1 systems possess multi-subunit crRNA-effector complexes, whereas in Class 2 systems all functions of the effector complex are carried out by a single protein (e.g., Cas9 or Cpf1). In some embodiments, the present disclosure provides using type II and/or type V single-subunit effector systems.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stern et al. (2010) Trends. Genet. 28: 335-340. For example, the Cse (Cas subtype, E. coli) proteins (e.g., CasA) form a functional complex, Cascade, which processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

(ii) CRISPR/Cas9

In some embodiments, the present disclosure provides methods of gene editing using a Type II CRISPR system. Type II systems rely on a i) single endonuclease protein, ii) a transactiving crRNA (tracrRNA), and iii) a crRNA where a ˜20-nucleotide (nt) portion of the 5′ end of crRNA is complementary to a target nucleic acid. The region of a CRISPR crRNA strand that is complementary to its target DNA protospacer is hereby referred to as “guide sequence.”

In some embodiments, the tracrRNA and crRNA components of a Type II system can be replaced by a single guide RNA (sgRNA), also known as a guide RNA (gRNA). The sgRNA can include, for example, a nucleotide sequence that comprises an at least 12-20 nucleotide sequence complementary to the target DNA sequence (guide sequence) and can include a common scaffold RNA sequence at its 3′ end. As used herein, “a common scaffold RNA” refers to any RNA sequence that mimics the tracrRNA sequence or any RNA sequences that function as a tracrRNA.

Cas9 endonucleases produce blunt end DNA breaks, and are recruited to target DNA by a combination of a crRNA and a tracrRNA oligos, which tether the endonuclease via complementary hybridization of the RNA CRISPR complex.

In some embodiments, DNA recognition by the crRNA/endonuclease complex requires additional complementary base-pairing with a protospacer adjacent motif (PAM) (e.g., 5′-NGG-3′) located in a 3′ portion of the target DNA, downstream from the target protospacer. (Jinek, M., et. al., Science. 2012, 337:816-821). In some embodiments, the PAM motif recognized by a Cas9 varies for different Cas9 proteins.

In some embodiments, the Cas9 disclosed herein can be any variant derived or isolated from any source. In other embodiments, the Cas9 peptide of the present disclosure can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27,156(5):935-49; Jinek M. et al. Science. 2012 337:816-21; and Jinek M. et al. Science. 2014 Mar. 14, 343(6176); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. Thus, in some embodiments, the systems and methods disclosed herein can be used with the wild type Cas9 protein having double-stranded nuclease activity, Cas9 mutants that act as single stranded nickases, or other mutants with modified nuclease activity.

According to the present disclosure, Cas9 molecules of, derived from, or based on the Cas9 proteins of a variety of species can be used in the methods and compositions described herein. For example, Cas9 molecules of, derived from, or based on, e.g., S. pyogenes, S. thermophilus, Staphylococcus aureus and/or Neisseria meningitidis Cas9 molecules, can be used in the systems, methods and compositions described herein. Additional Cas9 species include: Acidovorax avenae, Actinobacillus pleuropneumonias, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

In some embodiments, the present disclosure teaches the use of tools for genome editing techniques in plants such as crops and methods of gene editing using CRISPR-associated (cas) endonucleases including SpCas9, SaCas9, St1Cas9. These powerful tools for genome editing, which can be applied to plant genome editing are well known in the art. See example, Song et al. (2016), CRISPR/Cas9: A powerful tool for crop genome editing, The Crop Journal 4:75-82, Mali et al. (2013) RNA-guided human genome engineering via cas9, Science 339: 823-826; Ran et al. (2015) In vivo genome editing using Staphylococcus aureus cas9, Nature 520: 186-191; Esvelt et al. (2013) Orthogonal cas9 proteins for RNA-guided gene regulation and editing, Nature methods 10(11): 1116-1121, each of which is hereby incorporated by reference in its entirety for all purposes.

(iii) CRISPR/Cpf1

In other embodiments, the present disclosure provides methods of gene editing using a Type V CRISPR system. In some embodiments, the present disclosure provides methods of gene editing using CRISPR from Prevotella, Francisella, Acidaminococcus, Lachnospiraceae, and Moraxella (Cpf1).

The Cpf1 CRISPR systems of the present disclosure comprise 1) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA. In some embodiments, guide sequences for Cpf1 must be at least 12nt, 13nt, 14nt, 15nt, or 16nt in order to achieve detectable DNA cleavage, and a minimum of 14nt, 15nt, 16nt, 17nt, or 18nt to achieve efficient DNA cleavage.

The Cpf1 systems of the present disclosure differ from Cas9 in a variety of ways. First, unlike Cas9, Cpf1 does not require a separate tracrRNA for cleavage. In some embodiments, Cpf1 crRNAs can be as short as about 42-44 bases long—of which 23-25 nt is guide sequence and 19 nt is the constitutive direct repeat sequence. In contrast, the combined Cas9 tracrRNA and crRNA synthetic sequences can be about 100 bases long.

Second, certain Cpf1 systems prefer a “TTN” PAM motif that is located 5′ upstream of its target. This is in contrast to the “NGG” PAM motifs located on the 3′ of the target DNA for common Cas9 systems such as Streptococcus pyogenes Cas9. In some embodiments, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 bases, which create “sticky ends” (Kim et al., 2016. “Genome-wide analysis reveals specificities of Cpf1 endonucleases in human cells” published online Jun. 6, 2016). These sticky ends with 3-5 nt overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA.

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on Zetsche B. et al. 2015. (“Cpf1 Is a Single RNA-Guided Endonuclease of a Class 2 CRISPR-Cas System” Cell 163, 759-771).

(iv) Guide RNA (gRNA)

In some embodiments, the guide RNA of the present disclosure comprises two coding regions, encoding for crRNA and tracrRNA, respectively. In other embodiments, the guide RNA is a single guide RNA (sgRNA) synthetic crRNA/tracrRNA hybrid. In other embodiments, the guide RNA is a crRNA for a Cpf1 endonuclease.

Persons having skill in the art will appreciate that, unless otherwise noted, all references to a single guide RNA (sgRNA) in the present disclosure can be read as referring to a guide RNA (gRNA). Therefore, embodiments described in the present disclosure which refer to a single guide RNA (sgRNA) will also be understood to refer to a guide RNA (gRNA).

The guide RNA is designed so as to recruit the CRISPR endonuclease to a target DNA region. In some embodiments, the present disclosure teaches methods of identifying viable target CRISPR landing sites, and designing guide RNAs for targeting the sites. For example, in some embodiments, the present disclosure teaches algorithms designed to facilitate the identification of CRISPR landing sites within target DNA regions.

In some embodiments, the present disclosure teaches use of software programs designed to identify candidate CRISPR target sequences on both strands of an input DNA sequence based on desired guide sequence length and a CRISPR motif sequence (PAM, protospacer adjacent motif) for a specified CRISPR enzyme. For example, target sites for Cpf1 from Francisella novicida U112, with PAM sequences TTN, may be identified by searching for 5′-TTN-3′ both on the input sequence and on the reverse-complement of the input. The target sites for Cpf1 from Lachnospiraceae bacterium and Acidaminococcus sp., with PAM sequences TTTN, may be identified by searching for 5′-TTTN-3′ both on the input sequence and on the reverse complement of the input. Likewise, target sites for Cas9 of S. thermophilus CRISPR, with PAM sequence NNAGAAW, may be identified by searching for 5′-Nx-NNAGAAW-3′ both on the input sequence and on the reverse-complement of the input. The PAM sequence for Cas9 of S. pyogenes is 5′-NGG-3′.

Since multiple occurrences in the genome of the DNA target site may lead to nonspecific genome editing, after identifying all potential sites, sequences may be filtered out based on the number of times they appear in the relevant reference genome or modular CRISPR construct. For those CRISPR enzymes for which sequence specificity is determined by a ‘seed’ sequence (such as the first 5 bp of the guide sequence for Cpf1-mediated cleavage) the filtering step may also account for any seed sequence limitations.

In some embodiments, algorithmic tools can also identify potential off target sites for a particular guide sequence. For example, in some embodiments Cas-Offinder can be used to identify potential off target sites for Cpf1 (see Kim et al., 2016. “Genome-wide analysis reveals specificities of Cpf1 endonucleases in human cells” Nature Biotechnology 34, 863-868). Any other publicly available CRISPR design/identification tool may also be used, including for example the Zhang lab crispr.mit.edu tool (see Hsu, et al. 2013 “DNA targeting specificity of RNA guided Cas9 nucleases” Nature Biotech 31, 827-832).

In some embodiments, the user may be allowed to choose the length of the seed sequence. The user may also be allowed to specify the number of occurrences of the seed: PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s).

In the guide RNA, the “spacer/guide sequence” sequence is complementary to the “proto spacer” sequence in the DNA target. The gRNA “scaffold” for a single stranded gRNA structure is recognized by the Cas9 protein.

In some embodiments, the guide sequence is at least 15, 16, 17, 18, 19, 20, 21, 22, 23, 24, 25 or more nucleotides, or between 10-50, or between 15-45, or between 15-20, or between 20-35 or between 20-30 nucleotides in length. In some embodiments, the guide sequence is between 17-23 nucleotides in length.

In some embodiments, the plant, plant part, plant cell, or plant tissue culture taught in the present disclosure comprise a recombinant construct, which comprises at least one nucleic acid sequence encoding a guide RNA. In some embodiments, the nucleic acid is operably linked to a promoter. In other embodiments, a recombinant construct further comprises a nucleic acid sequence encoding a Clustered regularly interspaced short palindromic repeats (CRISPR) endonuclease. In other embodiments, the guide RNA is capable of forming a complex with said CRISPR endonuclease, and said complex is capable of binding to and creating a double strand break in a genomic target sequence of said plant genome. In other embodiments, the CRISPR endonuclease is Cas9.

In further embodiments, the target sequence is a nucleic acid for a gene, gene promoter, or genomic region associated with biotic and abiotic traits, such as pest and pathogen resistance, insect and disease resistance, nutrient biofortification and bioavailability, drought or flood tolerance, and developmental traits such as yield, growth height, seed size, flowering time, and hardiness.

In one embodiment, the present disclosure teaches the gene editing of phytoene desaturase (PDS) in plants using gene editing techniques described herein.

In some embodiments, the modified plant cells comprise one or more modifications (e.g., insertions, deletions, or mutations of one or more nucleic acids) in the genomic DNA sequence of an endogenous target gene resulting in the altered function the endogenous gene, thereby modulating, stimulating, or enhancing suberin content in plant cells, plant tissues, plant parts and whole plants. In such embodiments, the modified plant cells comprise a “modified endogenous target gene.”

In some embodiments, the modifications in the genomic DNA sequence cause mutation, thereby altering the function of the target protein associated with traits, such as pest and pathogen resistance, insect and disease resistance, nutrient biofortification, and drought or flood tolerance.

In some embodiments, the modifications in the genomic DNA sequence results in amino acid substitutions, thereby altering the normal function of the encoded protein. In some embodiments, the modifications in the genomic DNA sequence encode a modified endogenous protein with modulated, altered, stimulated or enhanced function compared to the unmodified version of the endogenous protein.

In some embodiments, the modified plant cells described herein comprise one or more modified endogenous target genes, wherein the one or more modifications result in an altered function of a gene product (i.e., a protein) encoded by the endogenous target gene compared to an unmodified plant cell. For example, in some embodiments, a modified plant cell demonstrates an downregulated expression of a protein or an upregulated expression of said protein.

In some embodiments, the expression of the gene product in a modified plant cell is enhanced by at least 0.5%, 1%, 2%, 3%, 4%, 5% or higher compared to the expression of the gene product in an unedited plant cell. In other embodiments, the expression of the gene product in a edited plant cell is enhanced by at least 10%, 20%, 30%, 40%, 50%, 60%, 70%, 80%, 90%, or more compared to the expression of the gene product.

In some embodiments, the expression of the gene product in a modified plant cell is reduced by at least 0.5%, 1%, 2%, 3%, 4%, 5% or higher compared to the expression of the gene product in an unedited plant cell. In other embodiments, the expression of the gene product in an edited plant cells reduced by at least 10%, 20%, 30%, 40%, 50%, 60%, 70%, 80%, 90%, or more compared to the expression of the gene product.

in an unedited plant cell. In some embodiments, the edited plant cells described herein demonstrate enhanced or reduced expression and/or function of gene products targeted by a plurality (e.g., two or more) of guide RNA (gRNA) compared to the expression of the gene products in an unedited plant cell. For example, in some embodiments, an edited plant cell demonstrates enhanced or reduced expression and/or function of gene products targeted by at least 2, 3, 4, 5, 6, 7, 8, 9, 10, or more gRNAs compared to the expression of the gene products in an unedited plant cell.

In some embodiments, the modified plant cells described herein comprise one or more modified endogenous target genes, wherein the one or more modifications to the target DNA sequence results in expression of a protein with enhanced or reduced or altered function (e.g., a “modified endogenous protein”) compared to the function of the corresponding protein expressed in an unmodified plant cell (e.g., a “unmodified endogenous protein”). In some embodiments, the modified plant cells described herein comprise at least 2, 3, 4, 5, 6, 7, 8, 9, 10, or more modified endogenous target genes encoding 2, 3, 4, 5, 6, 7, 8, 9, 10, or more modified endogenous proteins. In some embodiments, the modified endogenous protein demonstrates enhanced or altered binding affinity for another protein expressed by the modified plant cell or expressed by another cell; enhanced or altered signaling capacity; enhanced or altered enzymatic activity; enhanced or altered DNA-binding activity; or reduced or altered ability to function as a scaffolding protein.

In some embodiments, the CRISPR/Cas nuclease include Streptococcus pyogenes Cas9 (SpCas9) as well as Cas9 variants coming from other bacterial species: Staphylococcus aureus (SaCas9), Streptococcus thermophilus (StCas9) and Streptococcus canis (ScCas9). SpCas9 with the ‘NGG’ protospacer adjacent motif (PAM) is the most commonly used Cas9 variant for genome editing applications in various organisms, including plants. SaCas9 (‘NNGRRT’ PAM) and StCas9 (‘NNRGAA’ PAM) are successfully used in rice, tobacco and Arabidopsis. ScCas9, SpCas9-NG and SpCas9-derived xCas9 are characterized by broadened PAM motif requirements: ‘NNG’ for ScCas9, and ‘NG’ for SpCas9-NG and xCas9. We have also included modules with Cas12a (Cpf1) CRISPR nucleases from Francisella novicida (FnCas12a) and Lachnospiraceae bacterium (LbCas12a) as well as with four related Cms1 nucleases. LbCas12a, FnCas12a and Cms1 have all been shown to work in plants. The diverse source of CRISPR/Cas nucleases, their variants and orthologs are well known along with their uses in plants are well descried in Jinek et al. (2012), Kaya et al. (2016), Steinert et al. (2015), Wolter et al. (2018), Schmidt et al. (2019), Chatterjee et al. (2018), Nishmasu et al. (2018), Hu et al. (2018), Tang et al. (2017), Endo et al. (2016), Begemann et al. (2017), and Hahn et al. (2019), each of which is incorporated by reference in their entirety.

In some embodiments, the CRISPR/Cas nuclease of the present disclosure is Cas9 and its variants. Also, the present disclosure teaches that the Cas9 is derived from SpCas9, SaCas9, StCas9, or ScCas9. In some embodiments, the CRISPR/Cas nuclease of the present disclosure is plant codon-optimized SpCas9. The codon optimization for the Cas protein depend on a plant of interest for the gene editing. The codon optimization of Cas9 gene is applied according to the Codon Usage Database and graphical codon usage analyzer. In some embodiments, the codons of SpCas9 is modified to better match the host codon usage preferences without altering the resulting amino acid sequence.

Gene editing constructs of the present disclosure can be embodied in one or more expression cassettes containing one or more regulatory elements operably linked to nucleotide sequences encoding crRNAs for forming gRNAs that will hybridize to the target sequence(s) of the plant DNA, and the same or different one or more regulatory elements operably linked to nucleotide sequences encoding the plant optimized SpCas9 nuclease(s) of the present disclosure.

In embodiments, the expression cassette is constituted to express at least one, at least two, at least three, at least four, at least five or more gRNAs. In some embodiments, the expression cassette is constituted to express at least two gRNAs. In some embodiments, the expression cassette is constituted to express two gRNAs.

The present disclosure teaches a gene editing cloning system characterized by a plurality of expression cassettes comprising: (i) at least two guide RNAs (gRNAs), each of which comprises a guide sequence operably linked to a gRNA scaffold sequence wherein a stop signal is operably linked to the gRNA scaffold sequence; (ii) a gene encoding a CRISPR-associated (Cas) protein; and (iii) a selectable marker gene. In some embodiments, each expression cassette comprises a promoter. In some embodiments, the gene editing cloning system is utilized to edit a genome in a plant, a plant part or a plant cell thereof. In some embodiments, the gRNA comprises at least 15 nucleotides guide sequence complementary to a target gene sequence. In some embodiments, the guide sequence is at least 15, 16, 17, 18, 19, 20, 21, 22, 23, 24, 25 or more nucleotides, or between 10-50, or between 15-45, or between 15-20, or between 20-35 or between 20-30 nucleotides in length. In some embodiments, the guide sequence is between 17-23 nucleotides in length.

In some embodiments, the gRNA scaffold sequence is a nucleic acid sequence comprising SEQ ID NO: 2, or a sequence at least 90% identical thereto. In some embodiments, the gRNA scaffold sequence is a nucleic acid sequence at least 95%, 98%, or 99% identical to SEQ ID NO: 2. In some embodiments, the Cas protein is Cas9, which is plant-optimized. In some embodiments, the selectable marker gene is hygromycin phosphotransferase (HPTII) gene. In some embodiments, the stop signal is poly thymine (poly T).

In some embodiments, the target gene is edited. The present disclosure teaches that the editing of the target gene results in the downregulated, suppressed, reduced, or abolished expression of the target gene, thereby the reduced or abolished expression of protein encoding the target gene.

In some embodiments, the gRNA binds to at least one genomic region of a target gene, thereby acquiring or enhancing a trait selected from the group consisting of biotic and abiotic traits, such as pest and pathogen resistance, insect and disease resistance, nutrient biofortification and bioavailability, drought or flood tolerance, and developmental traits such as yield, growth height, seed size, flowering time, and hardiness.

### Golden Gate Cloning

Golden Gate Cloning is a technique used for molecular cloning which enables the simultaneous and directional assembly of multiple DNA fragments into a single piece in vitro, using Type IIS restriction enzymes and T4 DNA ligase. The commonly used Type IIS enzymes for this technique are BsaI, BsmBI, and BbsI. Unlike standard Type II restriction enzymes, these enzymes cut DNA outside of their recognition sites, creating non-palindromic overhangs. As there are 256 potential overhang sequences, different combinations of overhang sequences can be used to assemble multiple fragments of DNA, resulting in a typically scarless product. The final product does not have a recognition site for Type IIS restriction enzymes, making the reaction essentially irreversible. This golden gate cloning can be used to assemble many pieces of DNA simultaneously.

The present disclosure teaches use of Type ITS restriction enzymes such as BsaI and BsmBI to create carefully-designed overhangs and ligate the segments (e.g., expression cassettes) without scar sequences between them, thereby producing the quasi-scarless final construct, where the restriction enzyme sites remain on both sides of the insert. Additional segments can be inserted into the vectors without scars within an open reading frame.

The golden gate assembly can combine several constructs to make a multigene single construct using modular cloning system. Modular Cloning (MoClo) is a method that allows multiple DNA parts to be ligated together into a backbone in a single reaction, using Type IIS restriction sites. Modular Cloning is based on Golden Gate Assembly, which utilizes Type IIS restriction enzymes that cleave DNA outside of their recognition site on one side, allowing for the removal of restriction sites from the design and preventing the formation of excess base pairs or scars between DNA parts.

The present disclosure teaches this modular cloning that employs a set of 4-base pair fusion sites to ligate the DNA parts together, leaving 4-nucleotide scars from BbsI restriction enzyme between the parts in the final DNA sequence.

### Selectable Marker

The present disclosure teaches that the gene editing cloning system comprises a vector with a selectable/screenable marker. The selectable marker is a gene that, if expressed in plants or plant tissues, makes it possible to distinguish them from other plants or plant tissues that do not express that gene. Screening procedures may require assays for expression of proteins encoded by the screenable marker gene. Examples of such markers include the beta glucuronidase (GUS) gene and the luciferase (LUX) gene. The instant disclosure demonstrates that cyanamide tolerance genes such as CAH can also be used as a marker. Thus, a gene encoding resistance to a fertilizer, antibiotic, herbicide or toxic compound can be used to identify transformation events. Examples of selectable markers include the cyanamide hydratase gene (CAH) streptomycin phosphotransferase (SPT) gene encoding streptomycin resistance, the neomycin phosphotransferase (NPTII) gene encoding kanamycin and geneticin resistance, the hygromycin phosphotransferase (HPT or APHIV) gene encoding resistance to hygromycin, genes (amp and/or β-lactamase (bla)) encoding resistance to ampicillin, acetolactate synthase (als) genes encoding resistance to sulfonylurea-type herbicides, genes (BAR and/or PAT) coding for resistance to herbicides which act to inhibit the action of glutamine synthase such as phosphinothricin (Liberty or Basta), or other similar genes known in the art.

The marker genes corresponding to these various agents encode neomycin phosphotransferase (NPTII) for kanamycin or paramomycin resistance, hygromycin phosphotransferase (HPTII) for resistance to hygromycin, 5-enolpyruvul-3-phosphoshikimic acid synthase (EPSPS) for glyphosate resistance, phosphinothricin acetyltransferase (PAT) for glufosinate resistance, and cyanamide hydratase (CAH) for cyanamide resistance.

In some embodiments, the marker gene encodes hygromycin phosphotransferase (HPTII) for resistance to hygromycin. In some embodiments, the marker gene is the ampicillin resistance gene (amp or bla) encoding beta-lactamase for resistance to ampicillin.

### Promoters

A regulatory element in the gene editing system may be of any of various types, and may for example comprise one or more than one RNA polymerase III (Pol III) promoter. In some embodiments, the regulatory element in the gene editing system may include one or more promoters such as ZmUbi promoter, OsU6 promoter, OsU3 promoter, U6 promoter, or other suitable promoter or promoters. For example, a single RNA Poll III promoter may be employed in various gene editing systems of the present disclosure as a regulatory element for driving both the Cas9 endonuclease and the gRNA(s) expression in the system. In other embodiments, multiple ones of a same promoter may be employed for expression of two gRNAs. For example, dual or multiple Pol III promoter arrangements may be employed in the editing system. It will be recognized that numerous arrangements of regulatory elements may be employed in the gene editing systems of the present disclosure, in specific implementations thereof.

In some embodiments, the promoter for driving expression of gRNA is a rice OsU6-2 RNA polymerase III promoter, a rice U3 promoter, an Arabidopsis AtU6-26 promoter, a maize ZmU3 promoter, a soybean GmU6 promoter, or other RNA polymerase III promoters derived from plant species. In other embodiments, the promoter for driving expression of gRNA is a 35S promoter, 2× 35S promoter, an Arabidopsis AtUBQQ10 promoter, a maize ZmUBI promoter, or a rice UsACT1 promoter. In further embodiments of the present disclosure, the promoter for driving expression of gRNA is a rice OsU6-2 RNA polymerase III promoter. In some embodiments, the promoter for expression of the gene encoding the Cas protein is a 2× 35S Cauliflower mosaic virus (CaMV) promoter. In some embodiments, the promoter for expression of the selectable marker gene is a 35S Cauliflower mosaic virus (CaMV) promoter. Endogenously identified species-specific RNA Pol III promoters are well known in the art to enhance genome editing efficiency, such as Kor et al. (2023), which is incorporated by reference in its entirety.

In some embodiments, the OsU6-2 promoter sequence is SEQ ID NO: 24. In some embodiments, the promoter for driving expression of gRNA is a nucleic acid set forth in SEQ ID NO: 24 or a sequence at least 90% identical thereto. the promoter for driving expression of gRNA is a sequence at least 90% identical thereto acid sequence at least 90%, at least 91%, at least 92%, at least 93%, at least 94%, at least 95%, at least 96%, at least 97%, at least 98%, or at least 99% identical to SEQ ID NO: 24.

### Plants for Gene Editing

The present disclosure provides that one or more vectors described herein are used to produce a gene-edited plant. In some embodiments, the organism or subject is a plant. In certain embodiments, the organism or subject or plant is algae.

In one aspect, the disclosure provides for methods of modifying a target polynucleotide in a plant cell, which may be in vivo, ex vivo or in vitro. In some embodiments, the method comprises sampling a cell or population of cells from a plant, and modifying the cell or cells. Culturing may occur at any stage ex vivo. The cell or cells may even be re-introduced into the plant.

In one aspect, the disclosure provides for methods of modifying a target polynucleotide in a plant cell. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence.

In one aspect, the disclosure provides a method of modifying expression of a polynucleotide in a plant cell. In some embodiments, the method comprises allowing a CRISPR complex to bind to the polynucleotide such that said binding results in increased or decreased expression of said polynucleotide; wherein the CRISPR complex comprises a CRISPR enzyme complexed with a guide sequence hybridized to a target sequence within said polynucleotide, wherein said guide sequence is harbored by a guide RNA.

In the present disclosure, the plants as a subject of gene editing are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, black raspberry, blueberry, broccoli, Brussel's sprouts, cabbage, cane berry, canola, cantaloupe, carrot, cassava, cauliflower, cedar, a cereal, celery, chestnut, cherry, Chinese cabbage, citrus, Clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, peach, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, wild strawberry, yams, yew, and zucchini.

In some embodiments, the plants as a subject of gene editing are Acanthaceae, Achariaceae, Achatocarpaceae, Acoraceae, Acrobolbaceae, Actinidiaceae, Adelanthaceae, Adiantaceae, Adoxaceae, Aextoxicaceae, Aizoaceae, Akaniaceae, Alismataceae, Allisoniaceae, Alseuosmiaceae, Alstroemeriaceae, Altingiaceae, Alzateaceae, Amaranthaceae, Amaryllidaceae, Amblystegiaceae, Amborellaceae, Anacardiaceae, Anarthriaceae, Anastrophyllaceae, Ancistrocladaceae, Andreaeaceae, Aneuraceae, Anisophylleaceae, Annonaceae, Antheliaceae, Anthocerotaceae, Aphanopetalaceae, Aphloiaceae, Apiaceae, Apocynaceae, Apodanthaceae, Aponogetonaceae, Aquifoliaceae, Araceae, Araliaceae, Araucariaceae, Archidiaceae, Arecaceae, Argophyllaceae, Aristolochiaceae, Arnelliaceae, Asparagaceae, Aspleniaceae, Asteliaceae, Asteropeiaceae, Atherospermataceae, Aulacomniaceae, Austrobaileyaceae, Aytoniaceae, Balanopaceae, Balanophoraceae, Balantiopsaceae, Balsaminaceae, Barbeuiaceae, Barbeyaceae, Bartramiaceae, Basellaceae, Bataceae, Begoniaceae, Berberidaceae, Berberidopsidaceae, Betulaceae, Biebersteiniaceae, Bignoniaceae, Bixaceae, Blandfordiaceae, Blechnaceae, Bonnetiaceae, Boraginaceae, Boryaceae, Boweniaceae, Brachytheciaceae, Brassicaceae, Brevianthaceae, Bromeliaceae, Bruchiaceae, Brunelliaceae, Bruniaceae, Bryaceae, Bryobartramiaceae, Bryoxiphiaceae, Burmanniaceae, Burseraceae, Butomaceae, Buxaceae, Buxbaumiaceae, Byblidaceae, Cabombaceae, Cactaceae, Calceolariaceae, Calomniaceae, Calophyllaceae, Calycanthaceae, Calyceraceae, Calymperaceae, Calypogeiaceae, Campanulaceae, Campyneumataceae, Canellaceae, Cannabaceae, Cannaceae, Capparaceae, Caprifoliaceae, Cardiopteridaceae, Caricaceae, Carlemanniaceae, Caryocaraceae, Caryophyllaceae, Casuarinaceae, Catagoniaceae, Catoscopiaceae, Celastraceae, Centrolepidaceae, Centroplacaceae, Cephalotaceae, Cephalotaxaceae, Cephaloziaceae, Cephaloziellaceae, Ceratophyllaceae, Cercidiphyllaceae, Chaetophyllopsaceae, Chloranthaceae, Chonecoleaceae, Chrysobalanaceae, Cinclidotaceae, Circaeasteraceae, Cistaceae, Cleomaceae, Clethraceae, Cleveaceae, Climaciaceae, Clusiaceae, Colchicaceae, Columelliaceae, Combretaceae, Commelinaceae, Compositae, Connaraceae, Conocephalaceae, Convolvulaceae, Coriariaceae, Cornaceae, Corsiaceae, Corsiniaceae, Corynocarpaceae, Costaceae, Crassulaceae, Crossosomataceae, Cryphaeaceae, Crypteroniaceae, Ctenolophonaceae, Cucurbitaceae, Cunoniaceae, Cupressaceae, Curtisiaceae, Cyatheaceae, Cycadaceae, Cyclanthaceae, Cymodoceaceae, Cynomoriaceae, Cyperaceae, Cyrillaceae, Cyrtopodaceae, Cytinaceae, Daltoniaceae, Daphniphyllaceae, Dasypogonaceae, Datiscaceae, Davalliaceae, Degeneriaceae, Dendrocerotaceae, Dennstaedtiaceae, Diapensiaceae, Dichapetalaceae, Dicksoniaceae, Dicnemonaceae, Dicranaceae, Didiereaceae, Dilleniaceae, Dioncophyllaceae, Dioscoreaceae, Dipentodontaceae, Dipteridaceae, Dipterocarpaceae, Dirachmaceae, Disceliaceae, Ditrichaceae, Doryanthaceae, Droseraceae, Drosophyllaceae, Dryopteridaceae, Ebenaceae, Ecdeiocoleaceae, Echinodiaceae, Elaeagnaceae, Elaeocarpaceae, Elatinaceae, Emblingiaceae, Encalyptaceae, Entodontaceae, Ephedraceae, Ephemeraceae, Equisetaceae, Ericaceae, Eriocaulaceae, Erpodiaceae, Erythroxylaceae, Escalloniaceae, Eucommiaceae, Euphorbiaceae, Euphroniaceae, Eupomatiaceae, Eupteleaceae, Eustichiaceae, Exormothecaceae, Fabroniaceae, Fagaceae, Fissidentaceae, Flagellariaceae, Fontinalaceae, Fontinaliaceae, Fossombroniaceae, Fouquieriaceae, Frankeniaceae, Funariaceae, Garryaceae, Geissolomataceae, Gelsemiaceae, Gentianaceae, Geocalycaceae, Geraniaceae, Gesneriaceae, Gigaspermaceae, Ginkgoaceae, Gisekiaceae, Gleicheniaceae, Gnetaceae, Goebeliellaceae, Gomortegaceae, Goodeniaceae, Goupiaceae, Grammitidaceae, Grimmiaceae, Griseliniaceae, Grossulariaceae, Grubbiaceae, Gunneraceae, Gymnomitriaceae, Gyrostemonaceae, Haemodoraceae, Halophytaceae, Haloragaceae, Hamamelidaceae, Hanguanaceae, Haplomitriaceae, Haptanthaceae, Hedwigiaceae, Heliconiaceae, Helicophyllaceae, Helwingiaceae, Herbertaceae, Hernandiaceae, Himantandraceae, Hookeriaceae, Huaceae, Humiriaceae, Hydatellaceae, Hydnoraceae, Hydrangeaceae, Hydrocharitaceae, Hydroleaceae, Hydrostachyaceae, Hylocomiaceae, Hymenophyllaceae, Hymenophyllopsidaceae, Hymenophytaceae, Hypericaceae, Hypnaceae, Hypnodendraceae, Hypopterygiaceae, Hypoxidaceae, Icacinaceae, Iridaceae, Irvingiaceae, Isoetaceae, Iteaceae, Ixioliriaceae, Ixonanthaceae, Jackiellaceae, Joinvilleaceae, Jubulaceae, Jubulopsaceae, Juglandaceae, Juncaceae, Juncaginaceae, Jungermanniaceae, Kirkiaceae, Koeberliniaceae, Krameriaceae, Lacistemataceae, Lactoridaceae, Lamiaceae, Lanariaceae, Lardizabalaceae, Lauraceae, Lecythidaceae, Leguminosae, Lejeuneaceae, Lembophyllaceae, Lentibulariaceae, Lepicoleaceae, Lepidobotryaceae, Lepidolaenaceae, Lepidoziaceae, Leptodontaceae, Lepyrodontaceae, Leskeaceae, Leucodontaceae, Leucomiaceae, Liliaceae, Limeaceae, Limnanthaceae, Linaceae, Linderniaceae, Loasaceae, Loganiaceae, Lomariopsidaceae, Lophiocarpaceae, Lophocoleaceae, Lophoziaceae, Loranthaceae, Low iaceae, Loxsomataceae, Lunulariaceae, Lycopodiaceae, Lythraceae, Magnoliaceae, Makinoaceae, Malpighiaceae, Malvaceae, Marantaceae, Marattiaceae, Marcgraviaceae, Marchantiaceae, Marsileaceae, Martyniaceae, Mastigophoraceae, Matoniaceae, Mayacaceae, Meesiaceae, Melanthiaceae, Melastomataceae, Meliaceae, Melianthaceae, Menispermaceae, Menyanthaceae, Mesoptychiaceae, Metaxyaceae, Meteoriaceae, Metteniusaceae, Metzgeriaceae, Misodendraceae, Mitrastemonaceae, Mitteniaceae, Mniaceae, Molluginaceae, Monimiaceae, Monocarpaceae, Montiaceae, Montiniaceae, Moraceae, Moringaceae, Muntingiaceae, Musaceae, Myliaceae, Myodocarpaceae, Myricaceae, Myriniaceae, Myristicaceae, Myrothamnaceae, Myrtaceae, Myuriaceae, Nartheciaceae, Neckeraceae, Nelumbonaceae, Neotrichocoleaceae, Nepenthaceae, Neuradaceae, Nitrariaceae, Nothofagaceae, Notothyladaceae, Nyctaginaceae, Nymphaeaceae, Ochnaceae, Octoblepharaceae, Oedipodiaceae, Olacaceae, Oleaceae, Oleandraceae, Onagraceae, Oncothecaceae, Ophioglossaceae, Opiliaceae, Orchidaceae, Orobanchaceae, Orthorrhynchiaceae, Orthotrichaceae, Osmundaceae, Oxalidaceae, Oxymitraceae, Paeoniaceae, Pallaviciniaceae, Pandaceae, Pandanaceae, Papaveraceae, Paracryphiaceae, Passifloraceae, Paulowniaceae, Pedaliaceae, Pelliaceae, Penaeaceae, Pentadiplandraceae, Pentaphragmataceae, Pentaphylacaceae, Penthoraceae, Peraceae, Peridiscaceae, Petermanniaceae, Petrosaviaceae, Philesiaceae, Philydraceae, Phrymaceae, Phyllanthaceae, Phyllodrepaniaceae, Phyllogoniaceae, Phyllonomaceae, Physenaceae, Phytolaccaceae, Picramniaceae, Picrodendraceae, Pilotrichaceae, Pinaceae, Piperaceae, Pittosporaceae, Plagiochilaceae, Plagiogyriaceae, Plagiotheciaceae, Plantaginaceae, Platanaceae, Pleuroziaceae, Pleuroziopsaceae, Plocospermataceae, Plumbaginaceae, Poaceae, Podocarpaceae, Podostemaceae, Polemoniaceae, Polygalaceae, Polygonaceae, Polypodiaceae, Polytrichaceae, Pontederiaceae, Porellaceae, Portulacaceae, Posidoniaceae, Potamogetonaceae, Pottiaceae, Primulaceae, Prionodontaceae, Proteaceae, Pseudolepicoleaceae, Psilotaceae, Pteridaceae, Pterigynandraceae, Pterobryaceae, Ptilidiaceae, Ptychomitriaceae, Ptychomniaceae, Putranjivaceae, Quillajaceae, Racopilaceae, Radulaceae, Rafflesiaceae, Ranunculaceae, Rapateaceae, Regmatodontaceae, Resedaceae, Restionaceae, Rhabdodendraceae, Rhabdoweisiaceae, Rhachitheciaceae, Rhacocarpaceae, Rhamnaceae, Rhipogonaceae, Rhizogoniaceae, Rhizophoraceae, Ricciaceae, Riellaceae, Rigodiaceae, Roridulaceae, Rosaceae, Rousseaceae, Rubiaceae, Ruppiaceae, Rutaceae, Rutenbergiaceae, Sabiaceae, Salicaceae, Salvadoraceae, Salviniaceae, Santalaceae, Sapindaceae, Sapotaceae, Sarcobataceae, Sarcolaenaceae, Sarraceniaceae, Saururaceae, Saxifragaceae, Scapaniaceae, Scheuchzeriaceae, Schisandraceae, Schistochilaceae, Schistostegaceae, Schizaeaceae, Schlegeliaceae, Schoepfiaceae, Scorpidiaceae, Scrophulariaceae, Selaginellaceae, Seligeriaceae Sematophyllaceae, Serpotortellaceae, Setchellanthaceae, Simaroubaceae, Simmondsiaceae, Siparunaceae, Sladeniaceae, Smilacaceae, Solanaceae, Sphaerosepalaceae, Sphagnaceae, Sphenocleaceae, Spiridentaceae, Splachnaceae, Splachnobryaceae, Stachyuraceae, Staphyleaceae, Stegnospermataceae, Stemonaceae, Stemonuraceae, Stereophyllaceae, Stilbaceae, Strasburgeriaceae, Strelitziaceae, Stylidiaceae, Styracaceae, Surianaceae, Symplocaceae, Takakiaceae, Talinaceae, Tamaricaceae, Tapisciaceae, Targioniaceae, Taxaceae, Taxodiaceae, Tecophilaeaceae, Tetrachondraceae, Tetramelaceae, Tetrameristaceae, Tetraphidaceae, Thamnobryaceae, Theaceae, Theliaceae, Thelypteridaceae, Thomandersiaceae, Thuidiaceae, Thurniaceae, Thymelaeaceae, Ticodendraceae, Timmiaceae, Tofieldiaceae, Torricelliaceae, Tovariaceae, Trachypodaceae, Treubiaceae, Trichocoleaceae, Trigoniaceae, Triuridaceae, Trochodendraceae, Tropaeolaceae, Typhaceae, Ulmaceae, Urticaceae, Vahliaceae, Velloziaceae, Verbenaceae, Vetaformaceae, Violaceae, Vitaceae, Vittariaceae, Vivianiaceae, Vochysiaceae, Wardiaceae, Welwitschiaceae, Wiesnerellaceae, Winteraceae, Woodsiaceae, Xanthorrhoeaceae, Xeronemataceae, Xyridaceae, Zamiaceae, Zingiberaceae, and Zosteraceae.

In further embodiments, the plants as a subject of gene editing is Brassicas, Chenopodiaceae, Poacea, Fabaceae, Compositae, Cucurbitaceae, Convolvulaceae, Solanaceae, Annual crops, biennial crops, or perennial crops.

The methods for targeted gene-editing system as described herein can be used to confer desired traits on essentially any plant. In embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, grape, peach, pear, plum, raspberry, black raspberry, blackberry, cane berry, cherry, avocado, strawberry, wild strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). In some embodiments, fruit crops such as tomato, apple, peach, pear, plum, raspberry, black raspberry, blackberry, cane berry, cherry, avocado, strawberry, wild strawberry, grape and orange.

The present disclosure teaches that plants described above can be applied for the gene editing using the gene editing cloning system or kits described herein. In some embodiments, plants comprise orphan crops. In some embodiments, plants comprise of model species. In some embodiments, plants comprise of crops. In some embodiments, the orphan staple crop is cassava, cowpea, plantain, millet, sweet potato, sorghum, teff, or yam. A list of the orphan crops is found in Venezia et al. (2021), which is incorporated by reference in its entirety.

With recent advances in crop genomics, the ability to use CRISPR-Cas systems to perform efficient and cost effective gene editing and manipulation will allow the rapid selection and comparison of single and multiplexed genetic manipulations to transform such genomes for improved production and enhanced traits. In this regard reference is made to US patents and publications: U.S. Pat. No. 6,603,061—Agrobacterium-Mediated Plant Transformation Method; U.S. Pat. No. 7,868,149—Plant Genome Sequences and Uses Thereof and US 2009/0100536—Transgenic Plants with Enhanced Agronomic Traits, all the contents and disclosure of each of which are herein incorporated by reference in their entirety. In the practice of the disclosure, the contents and disclosure of Morrell et al “Crop genomics: advances and applications” Nat Rev Genet. 2011 Dec. 29; 13(2):85-96 are also herein incorporated by reference in their entirety.

In plants, pathogens are often host-specific. Plants have existing and induced defenses to resist most pathogens. Mutations and recombination events across plant generations lead to genetic variability that gives rise to susceptibility, especially as pathogens reproduce with more frequency than plants.

In plants, there can be non-host resistance, for example, the host and pathogen are incompatible. There can be partial resistance against all races of a pathogen, typically controlled by many genes, or can be complete resistance to some races of a pathogen but not to other races, typically controlled by a few genes. In a Gene-for-Gene level, plants and pathogens evolve together, and the genetic changes in one balance changes in other. Accordingly, using Natural Variability, breeders combine most useful genes for Yield, Quality, Uniformity, Hardiness, Resistance. Using the present disclosure, plant breeders or plant researchers can induce mutations using the gene editing cloning system and/or kits described herein.

For example, plant varieties having desired characteristics or traits employ the present disclosure to induce the rise of resistance genes, with more precision than previous mutagenic agents and hence accelerate and improve plant breeding programs even in orphan plants.

The present disclosure teaches a gene-edited or genetically engineered plant, a plant part, or a plant cell thereof, comprising the vector taught herein, wherein the target gene is edited. In some embodiments, the gene-edited or genetically engineered plant confers a trait selected from the group consisting of pest and pathogen resistance, insect resistance, plant-disease resistance, drought tolerance, flood tolerance, nutrient biofortification and high yield. In some embodiments, a tissue culture of cells produced from the plant taught herein comprise the vector of the present disclosure. In some embodiments, plant seeds are produced by growing the gene-edited or genetically engineered plant taught herein. In some embodiments, the target gene is edited in the seed and a plant or a plant part thereof is produced by growing the seed.

### Gene Editing Cloning Kits

The disclosure provides kits containing any one or more of the elements disclosed in the systems and methods taught herein. In some embodiments, the kit comprises a gene editing cloning system characterized by a plurality of expression cassettes comprising: (i) at least two guide RNAs (gRNAs), each of which comprise a guide sequence operably linked to a gRNA scaffold sequence and a termination signal; (ii) a gene encoding a CRISPR-associated (Cas) protein; and (iii) a selectable marker gene. In some embodiments, each expression cassette comprises a promoter. In some embodiments, the promoter for expression of each gRNA is a rice OsU6-2 promoter, an Arabidopsis AtU6-26 promoter, a maize Ubi promoter or an RNA polymerase III promoter. In some embodiments, the promoter for expression of the gene encoding the Cas protein is a 2× 35S Cauliflower mosaic virus (CaMV) promoter. In some embodiments, the promoter for expression of the selectable marker gene is a 35S Cauliflower mosaic virus (CaMV) promoter.

In some embodiments, the gene editing cloning system is utilized to edit a genome in a plant cell. In some embodiments, the gRNA comprises at least 15 nucleotides guide sequence complementary to a target gene sequence.

In some embodiments, the gRNA scaffold sequence is a nucleic acid sequence comprising SEQ ID NO: 2, or a sequence at least 90% identical thereto. In some embodiments, the gRNA scaffold sequence is a nucleic acid sequence at least 90%, at least 91%, at least 92%, at least 93%, at least 94%, at least 95%, at least 96%, at least 97%, at least 98%, or at least 99% identical to SEQ ID NO: 2.

In some embodiments, the gRNA scaffold sequence is present in SEQ ID NO: 14. In some embodiments, the gRNA scaffold sequence is present in a nucleic acid sequence at least 90%, at least 91%, at least 92%, at least 93%, at least 94%, at least 95%, at least 96%, at least 97%, at least 98%, or at least 99% identical to SEQ ID NO: 14. In further embodiments, SEQ ID NO: 14 comprises the gRNA scaffold sequence (SEQ ID NO: 2).

In some embodiments, the gRNA scaffold sequence is present in SEQ ID NO: 15. In some embodiments, the gRNA scaffold sequence is present in a nucleic acid sequence at least 90%, at least 91%, at least 92%, at least 93%, at least 94%, at least 95%, at least 96%, at least 97%, at least 98%, or at least 99% identical to SEQ ID NO: 15. In further embodiments, SEQ ID NO: 15 comprises the gRNA scaffold sequence (SEQ ID NO: 2).

In some embodiments, the Cas protein is Cas9. In some embodiments, the selectable marker gene is hygromycin phosphotransferase (HPTII) gene. In some embodiments, the gRNA binds to at least one target gene, thereby acquiring or enhancing a trait selected from the group consisting of pest and pathogen resistance, insect resistance, plant-disease resistance, drought tolerance, flood tolerance, nutrient biofortification and high yield. In some embodiments, the plant cell is derived from an orphan crop. In some embodiments, the orphan crop is cassava, cowpea, plantain, millet, sweet potato, sorghum, teff, or yam.

The present disclosure also teaches that a cloning kit comprises (i) at least two cloning vectors, each of which comprises a cassette comprising a promoter operably linked to a lacZ gene and a gRNA scaffold; (ii) a vector comprising a cassette comprising a gene encoding a CRISPR-associated (Cas) protein; (iii) a vector comprising a cassette comprising a first selectable marker; (iv) a destination vector comprising a cassette comprising a second selectable marker. In some embodiments, the lacZ gene in each of said two cloning vectors is replaced with a guide sequence complementary to a target gene sequence in a plant cell by a restriction-ligation reaction. In some embodiments, each cassette has unique overhangs at 5′ and 3′ ends for orderly assembly of multiple cassettes or fragments. In some embodiments, multiple cassettes comprising said two cassettes from (i), said cassette from (ii), and said cassette from (iii) are assembled into a destination vector backbone based on the unique overhangs. In some embodiments, the cassette from (iv) is relaced with the assembled multiple cassettes.

The present disclosure teaches that said cloning kit further comprises a premixed buffer, a first enzyme mix, a second enzyme mix, and at least one annealed primer pair as a control. In some embodiments, the gRNA scaffold sequence is a nucleic acid sequence comprising SEQ ID NO: 2, or a sequence at least 90% identical thereto. In some embodiments, the gRNA scaffold sequence is present in SEQ ID NO: 14, or a sequence at least 90% identical thereto. In some embodiments, the gRNA scaffold sequence is present in SEQ ID NO: 15, or a sequence at least 90% identical thereto.

In some embodiments, each of said at least two cloning vectors comprises a promoter for expression of each gRNA. In some embodiments, the first enzyme mix comprises BsaI type IIS restriction endonuclease. In some embodiments, the second enzyme mix comprises BbsI type IIS restriction endonuclease. In some embodiments, the second selectable marker gene is kanamycin resistance gene. In some embodiments, the editing of the target gene confers a trait selected from the group consisting of pest and pathogen resistance, insect resistance, plant-disease resistance, drought tolerance, flood tolerance, nutrient biofortification and high yield. In some embodiments, the plant cell is derived from an orphan crop.

Elements may be provided individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide RNA sequence for insertion into a vector so as to operably link the guide RNA sequence and a regulatory element.

In one aspect, the disclosure provides a kit comprising one or more of the components described herein. In some embodiments, the kit comprises a gene editing cloning system and instructions for using the kit. In some embodiments, the gene editing cloning system comprises (a) a first regulatory element operably linked to a guide sequence and a gRNA scaffold, wherein when expressed, the guide sequence directs sequence-specific binding of a CRISPR complex to a target sequence in a plant cell, wherein the CRISPR complex comprises a CRISPR associated effector complexed with (1) the guide sequence that is hybridized to the target sequence, and (2) the gRNA scaffold; and/or (b) a second regulatory element operably linked to an enzyme-coding sequence encoding said CRISPR associated effector. In some embodiments, the kit comprises components (a) and (b) located on the same or different vectors of the system.

In some embodiments, the gene editing cloning system comprises two of component (a), each of which comprises a different guide sequence under the control of the same first regulatory element. In other embodiments, component (a) further comprises two or more guide sequences operably linked to the first regulatory element, wherein when expressed, each of the two or more guide sequences direct sequence specific binding of a CRISPR complex to a different target sequence in a plant cell. In some embodiments, the system further comprises a third regulatory element operably linked to a selectable marker such as hygromycin phosphotransferase (HPTII) gene.

In some embodiments, the CRISPR-associated effector, protein, or enzyme comprises one or more nuclear localization sequences of sufficient strength to drive accumulation of said CRISPR-associated effector in a detectable amount in the nucleus of a plant cell. In some embodiments, the CRISPR-associated effector is a type II CRISPR system enzyme. In some embodiments, the CRISPR-associated effector is a Cas9 enzyme. In some embodiments, the Cas9 enzyme is S. pneumoniae, S. pyogenes or S. thermophilus Cas9, and may include mutated Cas9 derived from these organisms. The enzyme may be a Cas9 homolog or ortholog. In some embodiments, the CRISPR enzyme is codon-optimized for expression in a plant cell. In some embodiments, the CRISPR enzyme directs cleavage of one or two strands at the location of the target sequence. In some embodiments, the CRISPR enzyme lacks DNA strand cleavage activity. In some embodiments, the first regulatory element is a rice OsU6-2 promoter, an Arabidopsis AtU6-26 promoter, a maize Ubi promoter or an RNA polymerase III promoter. In some embodiments, the second regulatory element is a 35S or 2× 35S promoter. In some embodiments, the third regulatory element is a 35S or 2× 35S promoter.

The present disclosure teaches a cloning kit comprising: (i) at least two cloning vectors for gRNAs, each of which comprises a cassette comprising a promoter operably linked to a lacZ gene and a gRNA scaffold; (ii) a vector comprising a cassette comprising a gene encoding a CRISPR-associated (Cas) protein; (iii) a vector comprising a cassette comprising a first selectable marker; (iv) a destination vector comprising a second selectable marker. In some embodiments, the lacZ gene in each of said two cloning vectors is designed to be replaced with a guide sequence complementary to a target gene sequence in a plant cell by a restriction-ligation reaction. In some embodiments, each cassette has unique overhangs at 5′ and 3′ ends for orderly assembly of multiple cassettes or fragments. In some embodiments, multiple cassettes comprising said at least two cassettes from (i), said cassette from (ii), and said cassette from (iii) are assembled into a destination vector based on the unique overhangs. In some embodiments, the destination vector comprises the assembled multiple cassettes from (i), (ii), and (iii) vectors.

In some embodiments, the kit further comprises a premixed buffer, a first enzyme mix, a second enzyme mix, and at least one annealed primer pair as a control. In some embodiments of the kit, the gRNA scaffold sequence is a nucleic acid sequence comprising SEQ ID NO: 2, or a sequence at least 90% identical thereto. In some embodiments of the kit, each of said at least two cloning vectors comprises a promoter for expression of each gRNA. In some embodiments of the kit, the promoter is a rice OsU6-2 RNA polymerase III promoter. In some embodiments of the kit, the Cas protein is Cas9, which is plant-optimized. In some embodiments of the kit, the first selectable marker gene is hygromycin phosphotransferase (HPTII) gene. In some embodiments of the kit, the first enzyme mix comprises BsaI type IIS restriction endonuclease. In some embodiments of the kit, second enzyme mix comprises BbsI type IIS restriction endonuclease. In some embodiments of the kit, the second selectable marker gene is a red color selectable marker that is designed to be replaced with the assembled multiple cassettes. In some embodiments of the kit, the editing of the target gene confers a trait selected from the group consisting of pest and pathogen resistance, insect resistance, plant-disease resistance, drought tolerance, flood tolerance, nutrient biofortification and high yield. In some embodiments, a vector comprising the gene editing cloning system is provided.

### Methods for Modifying a Target Gene and Producing Gene-Edited Plants

The present disclosure provides methods for using one or more elements of a CRISPR/Cas9 system. The CRISPR complex of the disclosure provides an effective means for modifying a target polynucleotide. The CRISPR complex of the disclosure has a wide variety of utility including modifying (e.g., deleting, inserting, translocating, inactivating, activating) a target polynucleotide in a multiplicity of cell types. An exemplary CRISPR complex comprises a CRISPR enzyme complexed with a guide sequence hybridized to a target sequence within the target polynucleotide. A guide RNA comprises at least 15 nucleotides guide sequence.

The target polynucleotide of a CRISPR complex can be any polynucleotide endogenous or exogenous to the plant cell. For example, the target polynucleotide can be a polynucleotide residing in the nucleus of the plant cell. The target polynucleotide can be a sequence coding a gene product (e.g., a protein) or a non-coding sequence (e.g., a regulatory polynucleotide or a junk DNA). Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence) The skilled person will be able to identify PAM sequences for use with a given CRISPR enzyme.

The present disclosure provides a method of modifying a target polynucleotide in a plant cell. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a gRNA scaffold which in turn for a single guide RNA (sgRNA). In some embodiments, said cleavage comprises cleaving one or two strands at the location of the target sequence by said CRISPR enzyme. In some embodiments, said cleavage results in decreased transcription of a target gene. In some embodiments, the method further comprises repairing said cleaved target polynucleotide by homologous recombination with an exogenous template polynucleotide, wherein said repair results in a mutation comprising an insertion, deletion, or substitution of one or more nucleotides of said target polynucleotide. In some embodiments, said mutation results in one or more amino acid changes in a protein expressed from a gene comprising the target sequence. In some embodiments, the method further comprises delivering one or more vectors to said plant cell, wherein the one or more vectors drive expression of one or more of: the CRISPR enzyme and the single guide RNA. In some embodiments, said vectors are delivered to the plant cell in a plant. In some embodiments, said modifying takes place in said plant cell in a cell culture. In some embodiments, the method further comprises isolating said plant cell from a plant prior to said modifying. In some embodiments, the method further comprises regenerating said plant cell to make a mature plant.

The disclosure provides a method of modifying expression of a polynucleotide in a plant cell. In some embodiments, the method comprises allowing a CRISPR complex to bind to the polynucleotide such that said binding results in increased or decreased expression of said polynucleotide; wherein the CRISPR complex comprises a CRISPR enzyme complexed with a guide sequence hybridized to a target sequence within said polynucleotide, wherein said guide sequence is linked to a gRNA scaffold. In some embodiments, the method further comprises delivering one or more vectors to said plant cells, wherein the one or more vectors drive expression of one or more of: the CRISPR enzyme, the guide sequence linked to the gRNA scaffold.

The disclosure provides a method of generating a model plant cell comprising a mutated gene associated with a trait selected from the group consisting of pest and pathogen resistance, insect resistance, plant-disease resistance, drought tolerance, flood tolerance, nutrient biofortification and high yield. In some embodiments, the method comprises (a) introducing one or more vectors into a plant cell, wherein the one or more vectors drive expression of one or more of: a CRISPR enzyme, a guide sequence linked to a gRNA scaffold; and (b) allowing a CRISPR complex to bind to a target polynucleotide to effect cleavage of the target polynucleotide within said gene, wherein the CRISPR complex comprises the CRISPR enzyme complexed with (1) the guide sequence that is hybridized to the target sequence within the target polynucleotide, and (2) the gRNA scaffold, thereby generating a model plant cell comprising a mutated gene. In some embodiments, said cleavage comprises cleaving one or two strands at the location of the target sequence by said CRISPR enzyme. In some embodiments, said cleavage results in decreased transcription of a target gene. In some embodiments, the method further comprises repairing said cleaved target polynucleotide by homologous recombination with an exogenous template polynucleotide, wherein said repair results in a mutation comprising an insertion, deletion, or substitution of one or more nucleotides of said target polynucleotide. In some embodiments, said mutation results in one or more amino acid changes in a protein expression from a gene comprising the target sequence.

In one aspect, the disclosure provides a recombinant polynucleotide comprising a guide sequence upstream of a gRNA scaffold sequence, wherein the guide sequence when expressed directs sequence-specific binding of a CRISPR complex to a corresponding target sequence present in a plant cell. In some embodiments, the target sequence is a viral sequence present in a plant cell.

In one aspect the disclosure provides for a method of selecting one or more cell(s) by introducing one or more mutations in a gene in the one or more cell (s), the method comprising: introducing one or more vectors into the cell (s), wherein the one or more vectors drive expression of one or more of: a CRISPR enzyme, a guide sequence linked to a gRNA scaffold, and an editing template; wherein the editing template comprises the one or more mutations that abolish CRISPR enzyme cleavage; allowing homologous recombination of the editing template with the target polynucleotide in the cell(s) to be selected; allowing a CRISPR complex to bind to a target polynucleotide to effect cleavage of the target polynucleotide within said gene, wherein the CRISPR complex comprises the CRISPR enzyme complexed with (1) the guide sequence that is hybridized to the target sequence within the target polynucleotide, and (2) the gRNA scaffold, wherein binding of the CRISPR complex to the target polynucleotide induces cell death, thereby allowing one or more cell(s) in which one or more mutations have been introduced to be selected. In embodiments, the CRISPR enzyme is Cas9. In another embodiment of the disclosure the cell to be selected may be a plant cell. Aspects of the disclosure allow for selection of specific cells without requiring a selection marker or a two-step process that may include a counter-selection system.

The present disclosure provides a method for obtaining a cloning vector for expression of a gRNA of interest. In embodiments, the method comprises (a) preparing a gRNA primer pair with a overhang at 5′ end, wherein the primer pair is complementary to anneal a double stranded guide sequence molecule, and wherein 5′ end of each primer has the overhang; (b) digesting with an restriction enzyme a vector comprising a promoter operably linked to a lacZ gene which is operably linked to a gRNA scaffold and a termination/stop signal, wherein the lacZ gene is removed by the enzymatic reaction; (c) ligating the double stranded guide sequence molecule from (a) with the lacZ gene-depleted vector from (b); (d) obtaining a cloning vector that comprises an expression cassette comprising the promoter, the guide sequence, the gRNA scaffold, and the termination signal, wherein said expression cassette provide the gRNA of interest. In some embodiments, the cloning vector comprises at least two expression cassettes, each of which comprises the promoter, the guide sequence, the gRNA scaffold, and the termination signal. In some embodiments, the guide sequence at a 5′ end of each gRNA binds to a target gene. In some embodiments, the 5′ overhang is capable of ligating to BsaI restriction site. In some embodiments, the restriction enzyme is BsaI. In some embodiments, the target gene is edited, thereby its expression is downregulated, suppressed, reduced, or abolished. In some embodiments, a plant with the target gene edited confer a trait selected from the group consisting of pest and pathogen resistance, insect resistance, plant-disease resistance, drought tolerance, flood tolerance, nutrient biofortification and high yield.

The present disclosure also provides a method for producing a gene-edited plant in which a mutation is introduced into a specific target gene on a genome and no exogenous gene is incorporated on the genome. In embodiments, the method comprises (a) transforming the vector of the present disclosure into a plant cell; (b) culturing the plant cell obtained in the step (a) and selecting a regenerated plant; and (c) selecting a plant in which at least one target gene is edited by a CRISPR/Cas system. In some embodiments, the plant cell is derived from a monocot or a dicot. In further embodiments, the plant cell is derived from an orphan crop selected from a group consisting of cassava, cowpea, plantain, millet, sweet potato, sorghum, teff, and yam. In some embodiments, the vector comprises at least two gRNAs, each of which comprises at least 15 nucleotides guide sequence complementary to at least one genomic region of the target gene or functional derivative thereof. In some embodiments of the methods taught herein, said plant cell has one or more mutations in the genome which results in reduced or abolished expression of the target gene as compared to said expression in a normal cell that does not have such mutations. In some embodiments, the target gene is edited, thereby its expression is downregulated, suppressed, reduced, or abolished. In some embodiments, a plant with the target gene edited confer a trait selected from the group consisting of pest and pathogen resistance, insect resistance, plant-disease resistance, drought tolerance, flood tolerance, nutrient biofortification and high yield.

The present disclosure provides a cloning protocol for in planta CRISPR/Cas9 directed deletion. Necessary components for the gene editing cloning system are provided including promoters for both monocot and dicot expression, plant codon optimized Cas9 endonuclease, enzymes for Goldengate cloning, and antibiotic resistance markers for E. coli and agrobacterium, and herbicide selection. The two step cloning process that combines all vectors allows for the subcloning of two guide RNA into one complete binary vector for agro-mediated or biolistic bombardment in crops. To ensure the simplest cloning procedure of two guide RNA, and the ability to assess transformation efficiency, inventors developed vector plasmids described below, such as pGMP1 and pGMP2 for guide RNA positional subcloning, and another pGMP6 for eGFP nuclear localization in planta.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### Example 1. Generation of gRNA Subcloning Vectors

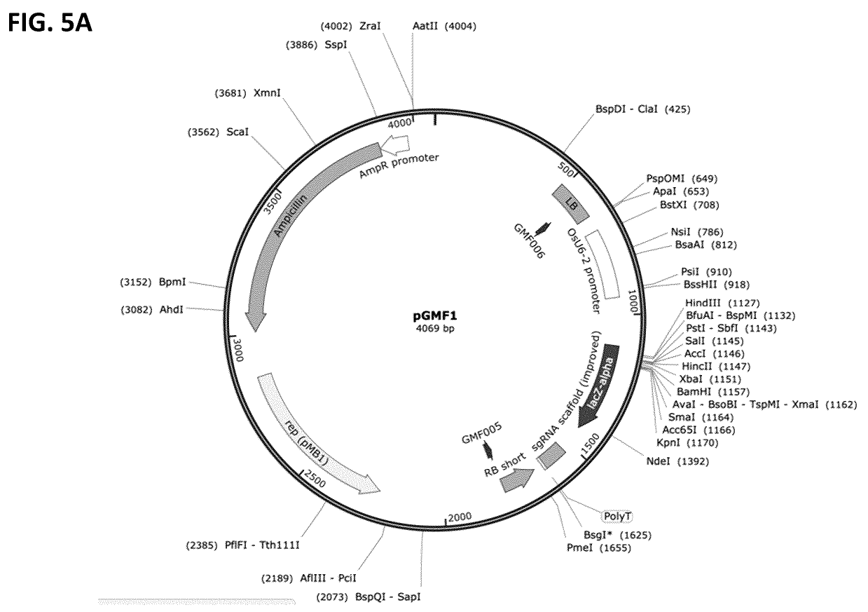

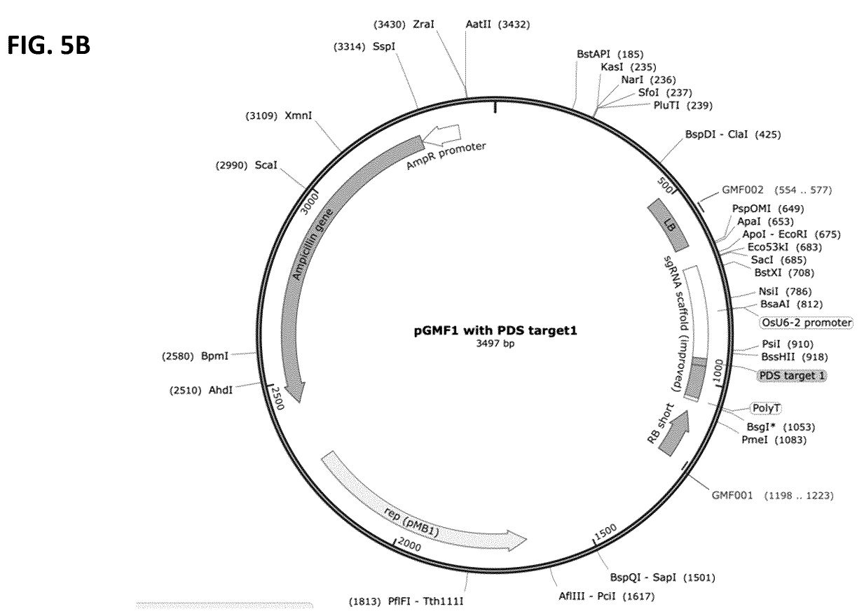

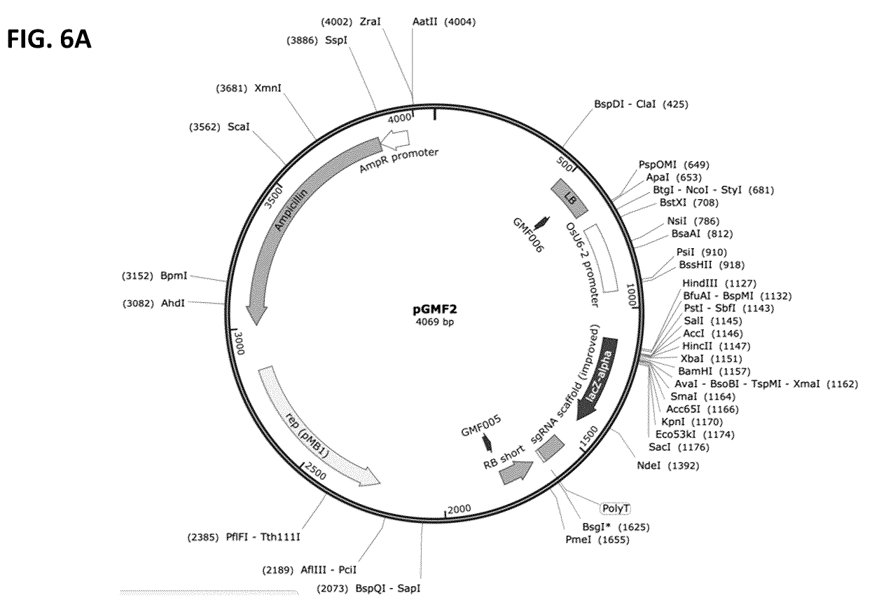

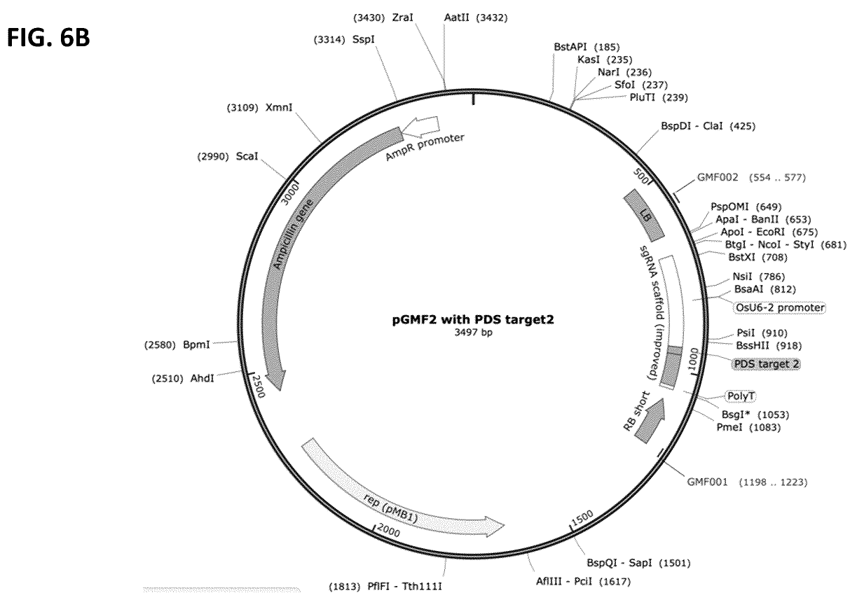

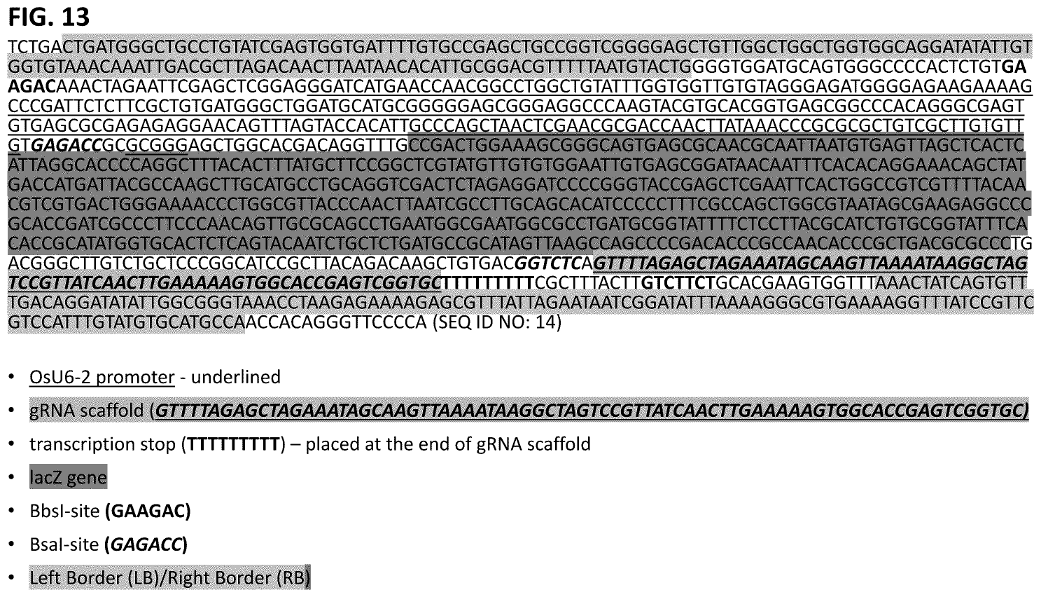

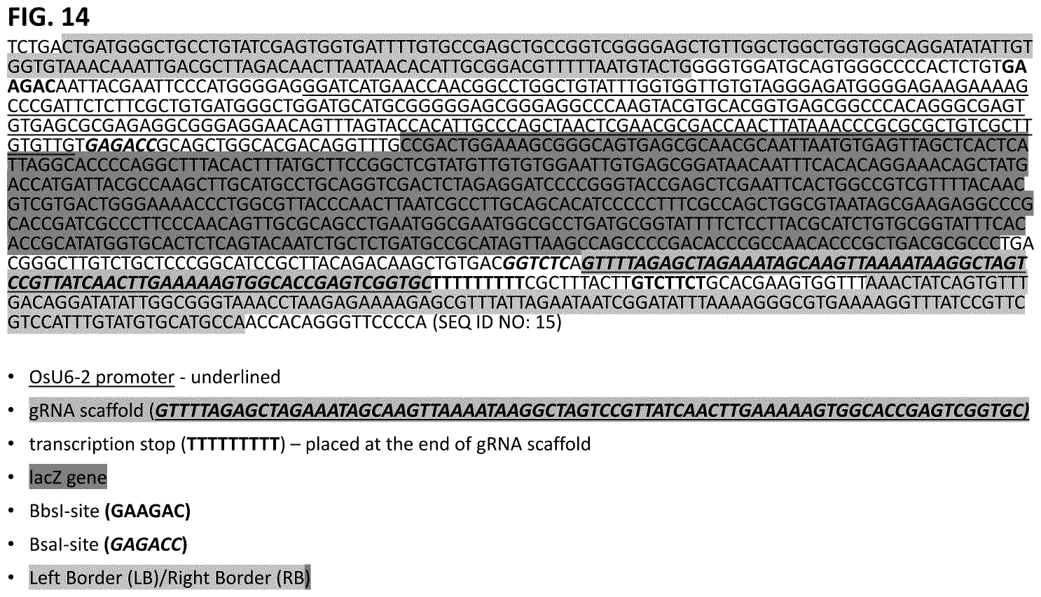

Inventors generated gRNA subcloning vectors by synthesizing expression cassettes (SEQ ID NO: 14 and SEQ ID NO: 15) and subcloning them into an Ampicillin resistant vector backbone. The gRNA subcloning vectors comprising SEQ ID NO: 14 (FIG. 13) and SEQ ID NO: 15 ((FIG. 14), are named as pGMP1 (FIG. 5A) and pGMP2 (FIG. 6A), respectively. Inventors introduced BbsI and BsaI restriction sites for golden gate cloning into pGMP1 and pGMP2 gRNA subcloning vectors. Also, rice U6-2 promoter was introduced to drive expression of gRNA that will be subcloned by swapping with LacZ that is a common screening cassette. LacZ is replaced by the multigene construct on the destination vector when gRNA of interest is added to the pGMP1 or pGMP2 vector. The BsaI restriction site will be lost once the LacZ gene is swapped with the guide sequence and a gRNA of interest is incorporated into pGMP1 and pGMP2 vectors. The gRNA scaffold sequence (SEQ ID NO: 2) is present in the expression cassette and operably linked to LacZ (which will be replaced with about 20 nt of gRNA sequence) and a stop signal (poly T sequence).

### Example 2. Designing Guide RNA Annealed Primer Pairs

The gene editing cloning system of the present disclosure is designed for the cloning of two guide RNAs for expression in plants. Target gene(s) can be identified by an interest or need of an user of the gene editing cloning system. Design and selection of gRNA binding to the target gene(s) can be carried out by freely available online resources.

If at least two targets in gene of interest are identified and selected, a vector comprising at least two gRNAs for the knocking out of the target gene can be generated by the gene editing cloning system of the present disclosure. The target should be 20 bp long and needs to be followed by the protospacer adjacent motive NGG.
Target 1: N20 NGG
Target 2: N20 NGG

When identifying the target site, it should not contain a BbsI (GAAGAC) and BsaI (GGTCTC) recognition sites as this will interfere with GG cloning. It is recommended that GC content of the 20 bp target site between 25%-75%, no more than 3 T in a row (long T stretches serve as stop signal for Polymerase III), good secondary structure of guide with intact stem loops.

Complementary primers for target sites with overhangs for cloning can be prepared and synthesized like below.
Annealed primer pair for target 1: gttgN20 and aaac N20 reverse complement
Annealed primer pair for target 2: gttgN20 and aaac N20 reverse complement

Further detailed steps for designing guide RNA annealed primer pairs are described below.

Step 1: Designing and validating gRNA with high efficiency and minimal off target activity. It is recommended to avoid designing gRNA in untranslated regions, introns, intergenic regions, and intron-exon junctions. It is suggested to focus on potential gRNA in exon order for higher likelihood of functional knockout.
i. Determine 5′ to 3′ target gene of interest DNA sequence and align multiple gene copies (if existing) to determine consensus sequence for targeting all.
ii. Determine exonic and intronic regions. If yet to be determined through RNA sequencing, utilize prediction algorithms for exonic regions.
iii. Highlight all NGG protospacer adjacent motif (PAM) sites within exonic sequences. If utilizing an online program, copy/paste exon(s) for gRNA candidates. Make sure to select the NGG PAM site, SpCas9, and your chosen reference genome.
iv. Select five-ten potential 20 bp gRNA candidates avoiding those with:
   Off targets with fewer than three mismatches and no mismatches within the seed region (8-12 bp adjacent to PAM)
   Poly-T stretches that serve as stop signal for Polymerase III
   Containing restriction sites BsaI (5′ GGTCTC . . . 3′) or BbsI (5′ GAAGAC . . . 3′) that will interfere with Golden Gate cloning
   A GC content less than 25% or more than 75%
v. Add each potential gRNA sequence to the full sequence below, assessing good secondary structure by minimum free energy and ensuring it contains 3 stem loops. Phytoene desaturase (PDS) gene in banana and plantain (Musa spp.) was selected as a target gene. SEQ ID NO: 1 is one example of the guide sequence, which targets PDS gene in Musa spp., operably linked to the gRNA scaffold sequence (SEQ ID NO: 2) and a stop signal (Poly-T region). Robust CRISPR/Cas9 mediated genome editing tool for banana and plantain (Musa spp.) is known in Ntui et al. (2020), which is incorporated by reference in its entirety.

guide sequence + Scaffold + Poly-T region
(SEQ ID NO: 1)
GTATCAATGATCGCTTGCAAGTTTCAGAGCTATGCTGGAAACAGCATAG
CAAGTTGAAATAAGGCTAGTCCGTTATCAACTTGAAAAAGTGGCACCGA
GTCGGTGCTTTTTTTTT
Scaffold
(SEQ ID NO: 2)
GTTTCAGAGCTATGCTGGAAACAGCATAGCAAGTTGAAATAAGGCTAGT
CCGTTATCAACTTGAAAAAGTGGCACCGAGTCGGTGC

Step 2: Design and obtain four complementary primer pairs with overhangs (two per gRNA).

It is important to know that the PAM site is not part of the gRNA and therefore not included in the primer pairs with overhangs.
i. Add GTTGN1 . . . N20 to the forward and AAACN20 . . . N1 to the reverse complement of gRNA1. (see example below)
ii. Add GTTGN1 . . . N20 to the forward and AAACN20 . . . N1 to the reverse complement of gRNA2. (see example below)
iii. Obtain four synthesized oligos (two annealed primer pairs per gRNA).
iv. Dilute to working concentration of 10 μM.

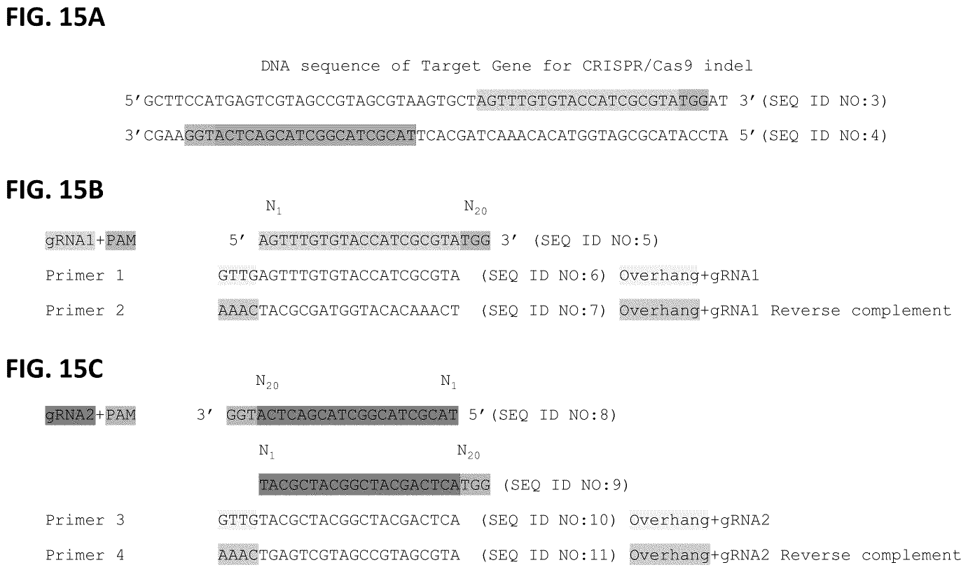

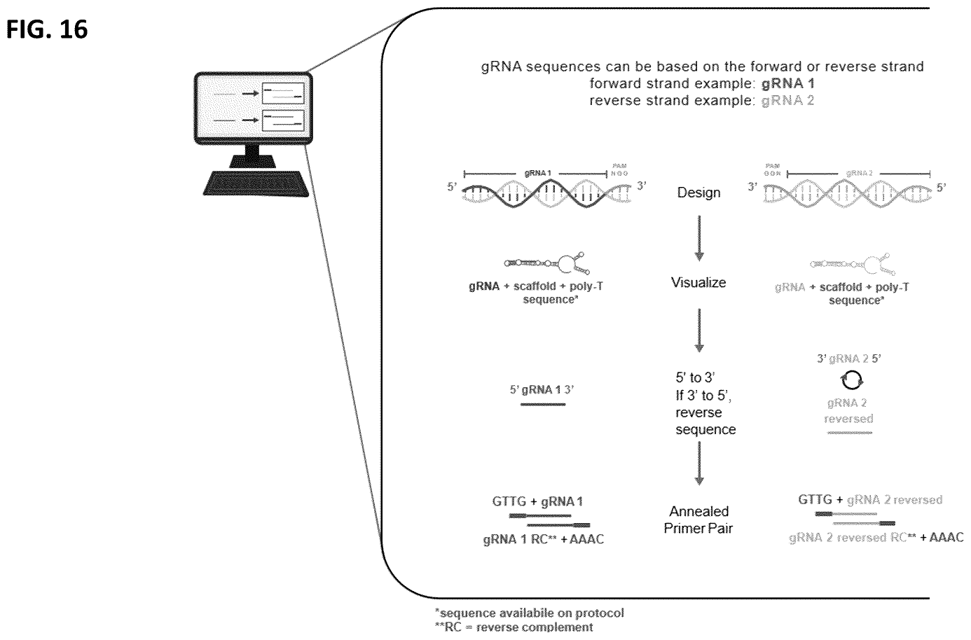

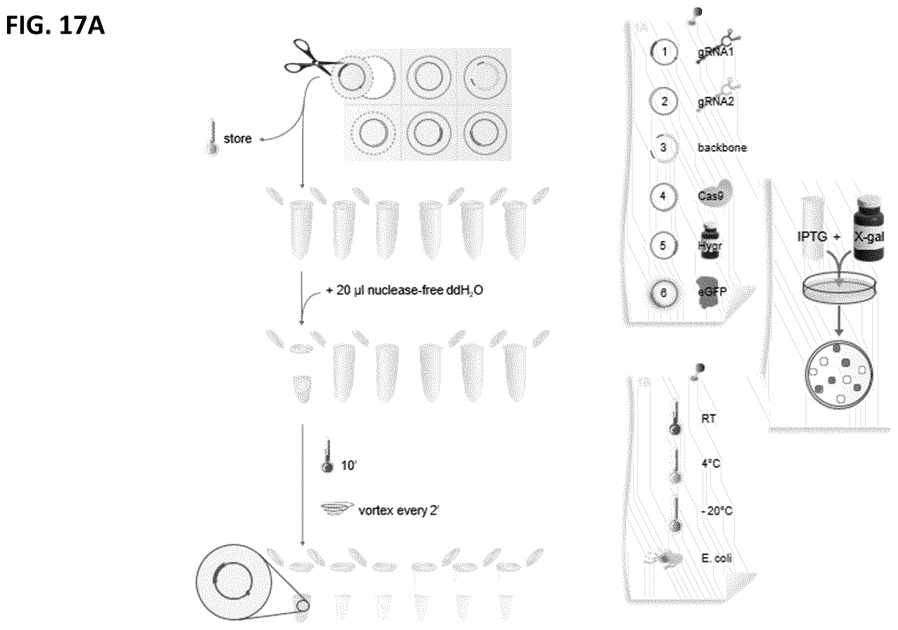

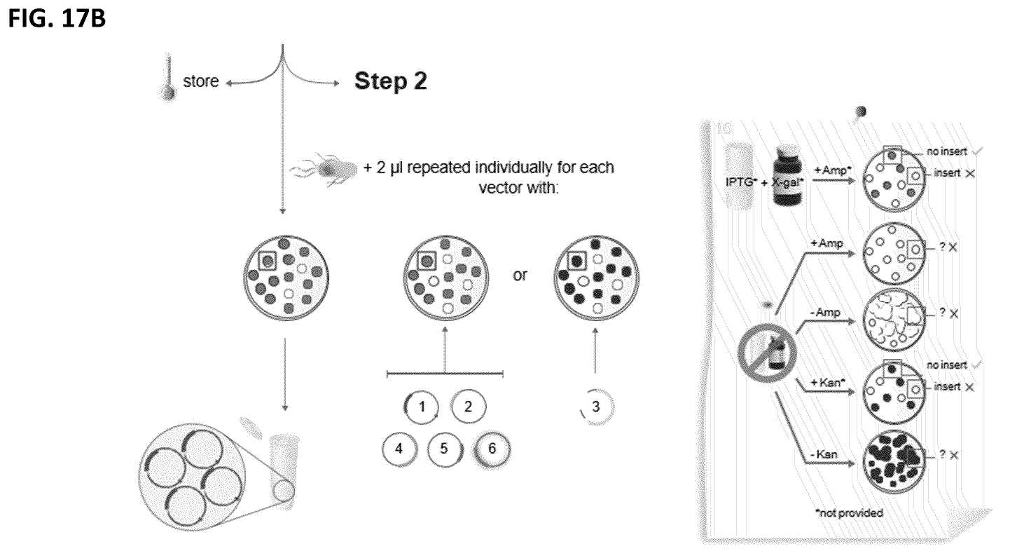

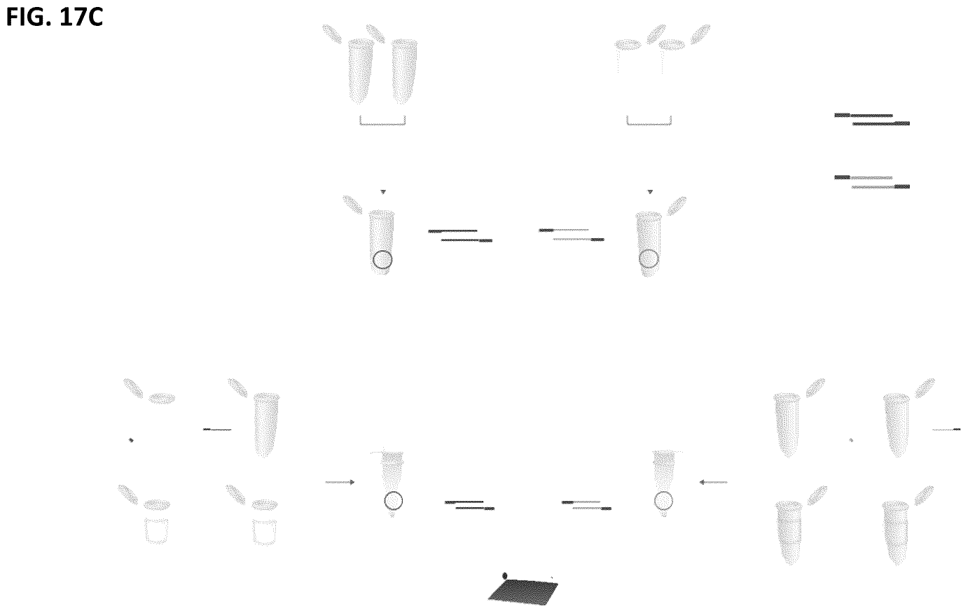

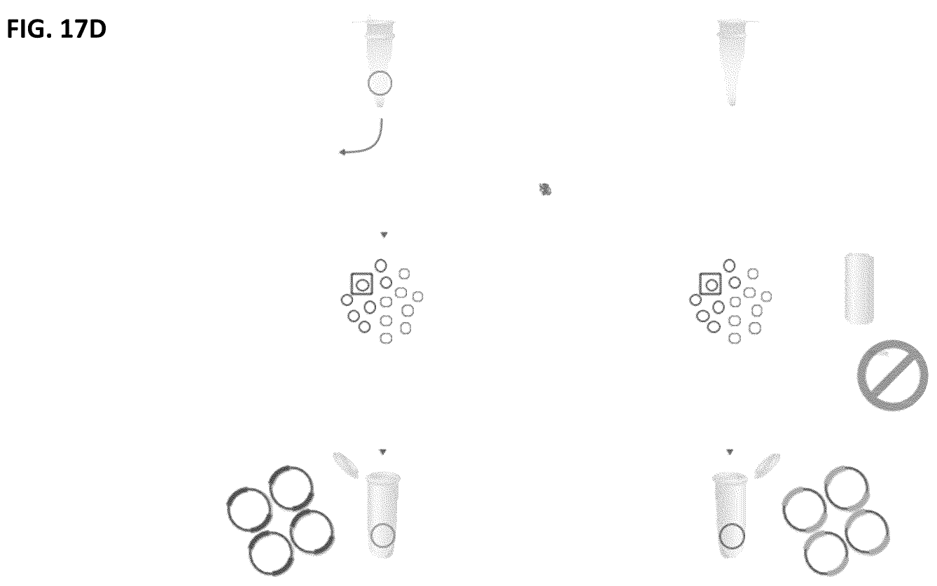

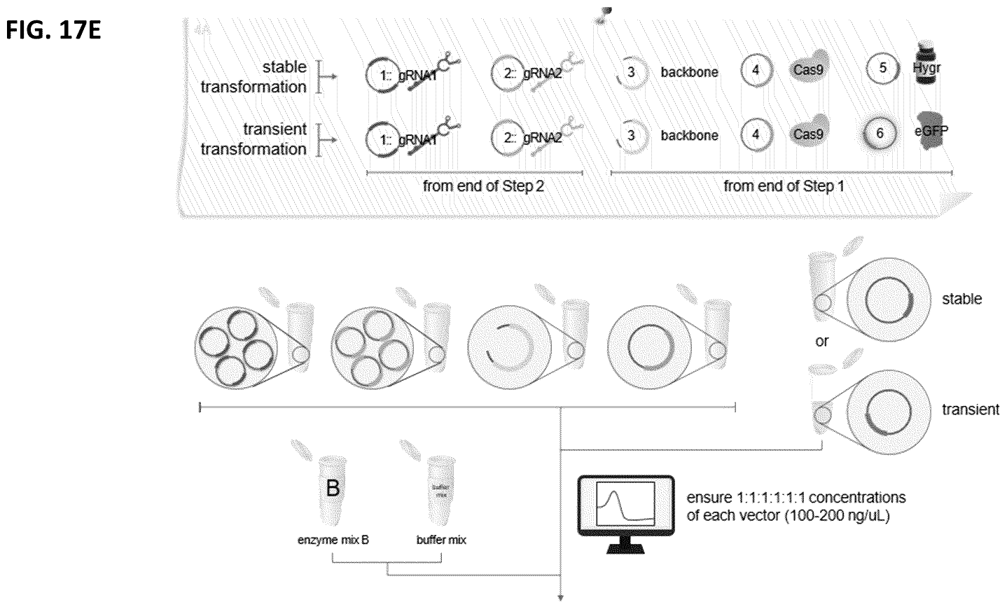

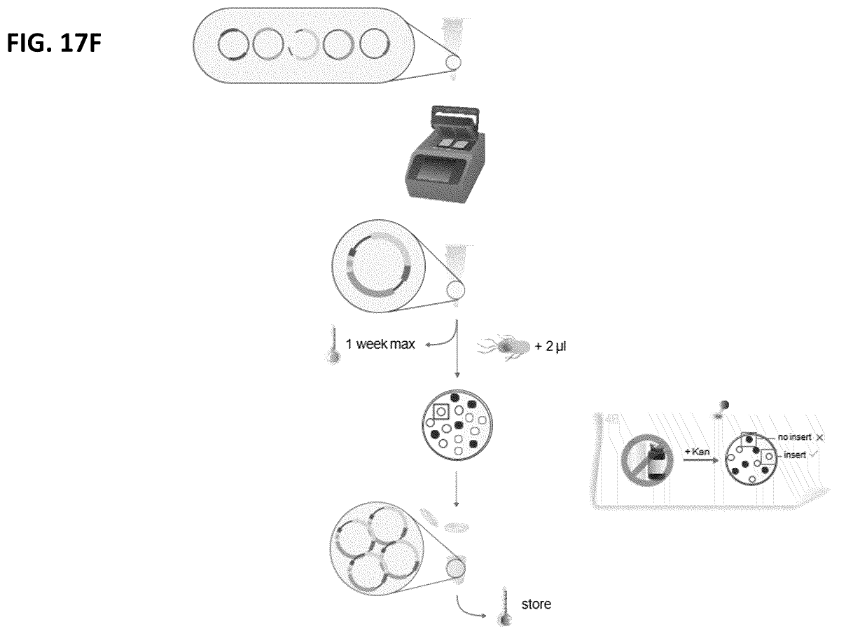

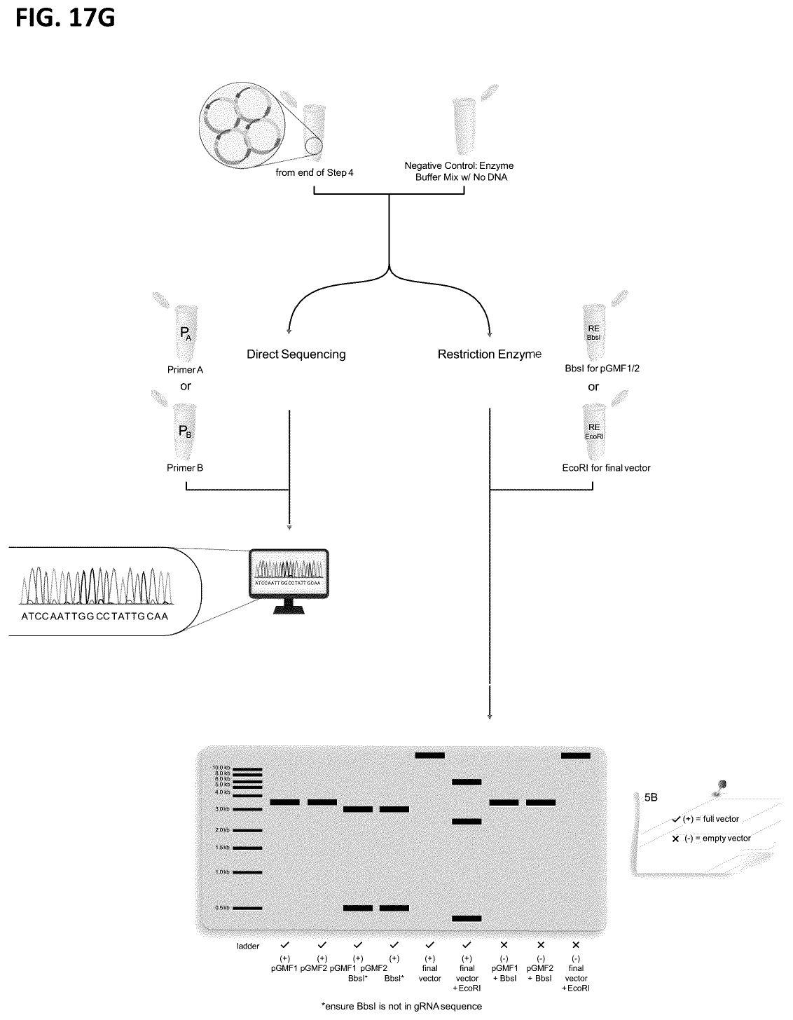

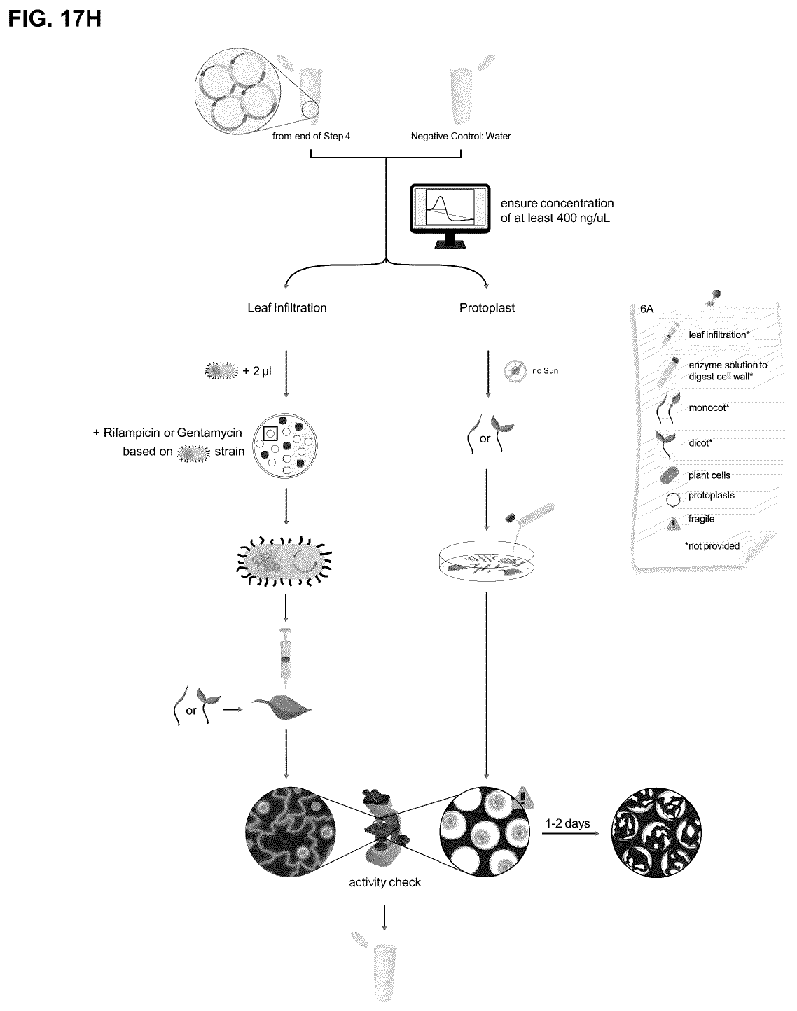

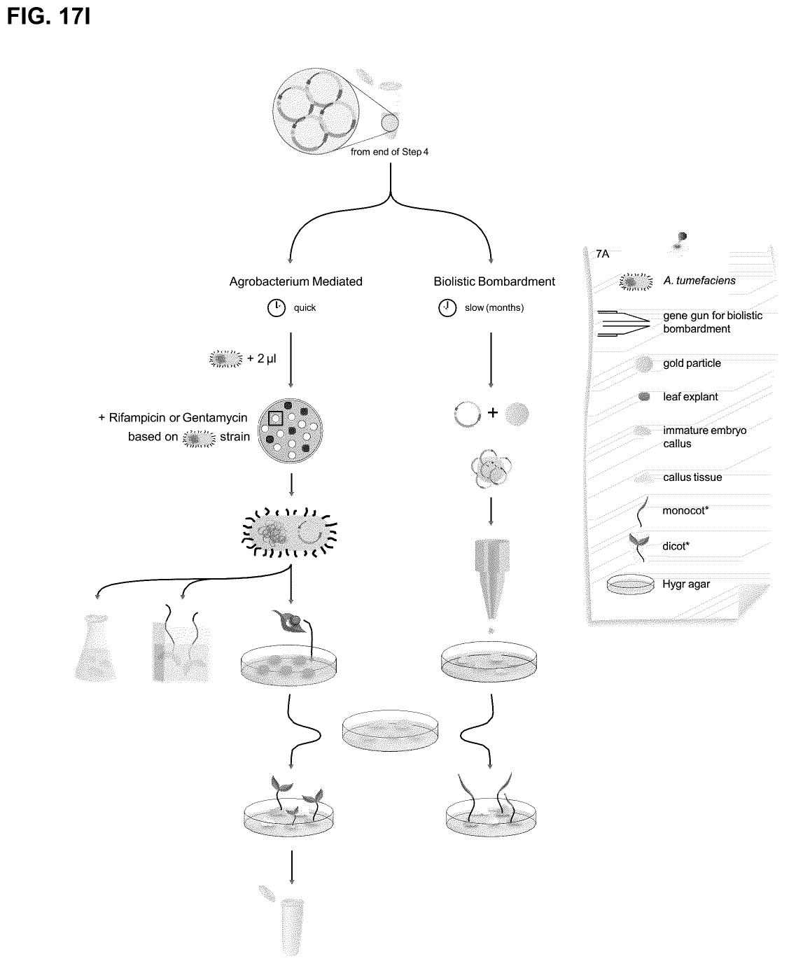

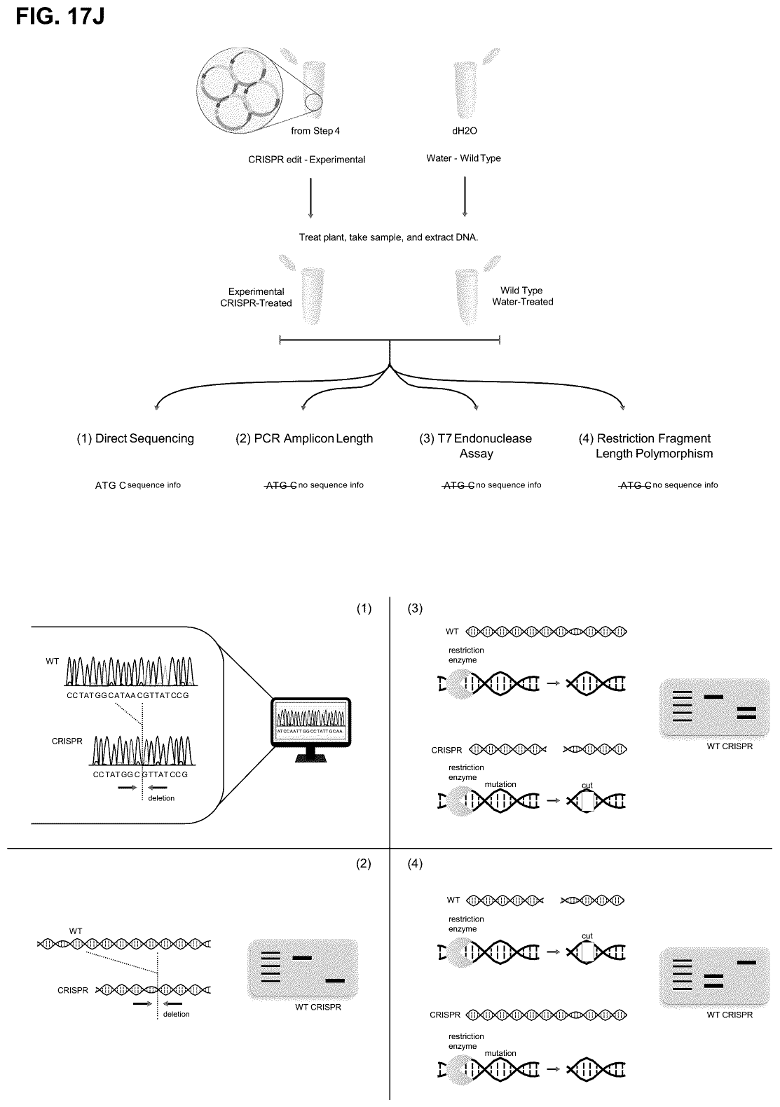

The example is given in FIGS. 15A-15C for gRNA on both forward and reverse target DNA sequence. The overhangs are not added to the 3′ PAM site but read as 5′ to 3′.

### Example 3. Subcloning Guide RNA Sequences

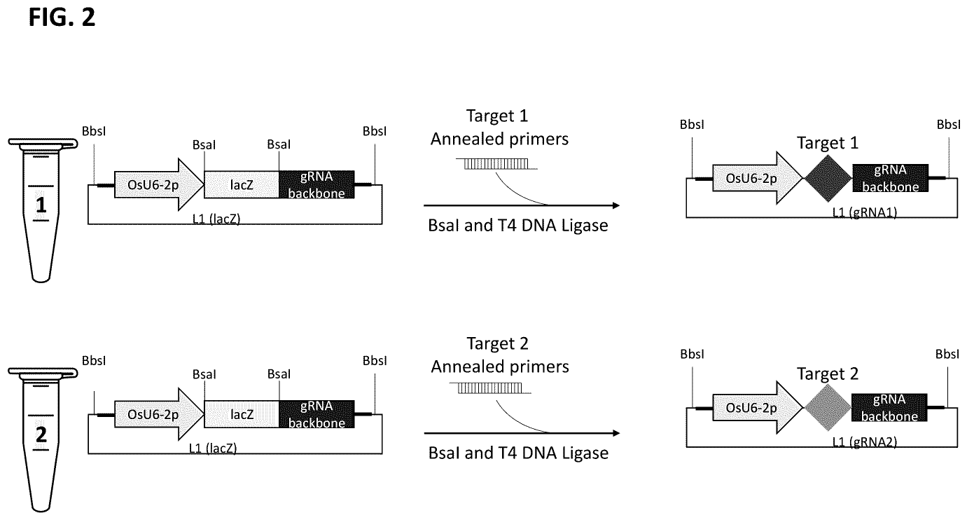

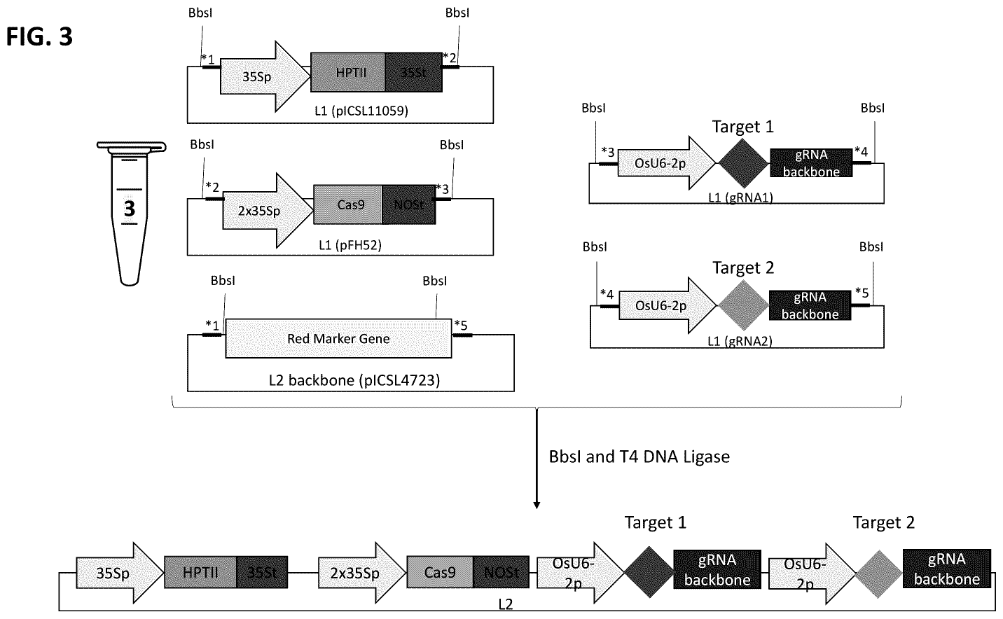

The gene editing cloning system of the present disclosure is designed for cloning two guide RNAs. Accordingly, gRNA annealed primer pairs are required. The gRNA annealed primer pairs can be prepared as described in Example 2. After designing four complementary primer pairs with overhangs (two per gRNA), each primer pairs are annealed for subcloning into a pGMF vector as presented in FIG. 2.

Control annealed primer pairs and control plasmid were prepared and tested for the gene editing cloning system. For the control of the gene editing cloning system of the present disclosure, phytoene desaturase (PDS) gene in banana and plantain (Musa spp.) was selected as a target gene. For the efficient CRISPR/Cas9 genome editing protocol, two gRNAs targeting phytoene desaturase (PDS) gene were selected using as described in Example 2. The last three bases in bold (TGG for Target 1 and GGG for Target 2) are the protospacer adjacent motif (PAM) NGG. This motif needs to be present next to the 20 bp gRNA target site. It is not part of the guide RNA itself though and is therefore not included in the primer sequences provided below

Target 1:
(SEQ ID NO: 16)
GTATCAATGATCGCTTGCAA TGG
Target 2:
(SEQ ID NO: 17)
TTTTGCCAGCCATGCTTGGA GGG

Complementary primers for target sites with the following 4 bp overhangs for cloning were generated.
Annealed primer pair for target 1: gttgGTATCAATGATCGCTTGCAA (Forward primer: SEQ ID NO: 18) and aaacTTGCAAGCGATCATTGATAC (Reverse primer: SEQ ID NO: 19)
Annealed primer pair for target 2: gttgTTTTGCCAGCCATGCTTGGA (Forward primer: SEQ ID NO: 20) and aaacGTATCAATGATCGCTTGCAA (Reverse primer: SEQ ID NO: 21)

Primer concentrations were adjusted to 10 μM. The forward and reverse primers were mixed in a 1:1 ratio and incubated for 5 min at room temperature. The annealed primers were integrated into the two different Level 1 (L1) subcloning vectors (i.e., pGMP1 and pGMP2) using two GG cut-ligation reactions via BsaI-HFv2. 2 μL of the GG reaction was transformed into competent cells and plate the cells on selection plates containing carbenicillin, IPTG and X-Gal. A white colony (negative colonies are blue) was inoculated and plasmid DNA was prepared. Correct integration of the annealed primer was verified by sequencing with primer Lvl1_F(0229) (GAACCCTGTGGTTGGCATGCACATAC; SEQ ID NO: 22) and Lvl1_R (0230) CTGGTGGCAGGATATATTGTGGTG (SEQ ID NO: 23). The L1 sgRNA constructs were combined with a L1 HPTII resistance marker transcription unit (pGMF5), a Cas9 L1 module (pGMF4) into the L2 backbone (pGMF3) using a GG cut-ligation reaction via BbsI-HF. 2 μL of the GG reaction was transformed into competent cells and plate the cells on kanamycin plates. A white colony (negative colonies are pink) was inoculated, plasmid DNA was prepared and correct assembly was verified by suitable restriction digest and sequencing.

gRNA target site selection can be done in any plant species if their genome information is publicly available and accessible. Once the target site is identified, annealed primer pairs can be prepared as described above and subcloned into pGMP1 and pGMP2 vectors to make a final vector for knockout of the target gene in the plant of interest.

### Example 4. Gene Editing Protocol for CRISPR/Cas9 Knockout in Planta

The gene editing cloning system is designed for cloning two guide RNA. The protocol for designing gRNA annealed primer pairs is provided in Examples 2 and 3. Once pGMF1 and pGMF2 subcloning vectors are ready by swapping LacZ gene with the gRNAs of interest, the following steps can follow for CRISPR/Cas9 Knockout of the target gene(s) in plants of interest.

Step 1: Recovering pGMF plasmids (i.e., from pGMF1 to pGMF6 vectors).
i. Cut each filter paper disk(s) using a sterile blade and place separately into labeled tube(s)
ii. Add 20 μl of double-distilled nuclease-free water.
iii. Incubate for 10 minutes at room temperature and vortex/mix every 2 minutes.
Optional: Transform 2 μl of each elution into competent E. coli.
iv. Proceed to Step 2.
v. Store any remaining eluted plasmid DNA at −20° C.

Step 2: Subcloning two guide RNA. Set up two independent restriction-ligation reactions for each set of annealed primer pairs per gRNA. One tube containing pGMF1::gRNA1 annealed primer pair 1 and the other pGMF2::gRNA2 annealed primer pair 2.
i. Adjust primer concentration to 10 μM, mix 2 μl of both annealed primer pairs per gRNA and incubate for 5 minutes at room temperature.

Optional: Control gRNA annealed primer pairs are adjusted.
ii. Set up the following two restriction-ligation reactions:
   1 μl pGMF1 or pGMF2 (100 ng/μl)
   1 μl Annealed Primer Pair 1 or 2 (10 μM)
   11 μl Premixed Buffer 1
   2 μl Enzyme mix A
iii. Run the following thermocycler program for restriction-ligation reactions and if not immediately transforming, store at 4° C. for up to one week if not immediately transforming into competent E. coli. Depending on thermocycler, this program can run 7+ hours.
   1. First Restriction 37° C. 20 seconds
   2. Restriction 37° C. 3 minutes
   3. Ligation 16° C. 4 minutes
   4. Repeat Steps 2-3 50×
   5. First heat inactivation 50° C. 5 minutes
   6. Final heat inactivation 80° C. 5 minutes
   7. Hold 16° C.
   8. Terminal Hold 16° C. *If running overnight
iv. Transform 2 μl of each restriction-ligation reaction separately in two aliquots of competent E. coli. Grow on Ampicillin antibiotic resistance, select, and extract plasmid DNA from white E. coli colonies on with IPTG/X-gal blue-white selection.

Step 3: Cloning the full construct for subsequent plant transformation by A. tumefaciens or biolistic bombardment. Important to note that Step 3.i. combines both plasmids extracted from Step 2.iv. into one final restriction-ligation reaction. pGMF5 vector can be substituted with pGMF6 for transient activity in cells.
i. Set up the following restriction-ligation reaction:
   1 μl pGMF1::gRNA1 (100 ng/μl)
   1 μl pGMF2::gRNA2 (100 ng/μl)
   1 μl per plasmid pGMF3, pGMF4, and pGMF5 (100 ng/μl)
   8 μl Premixed Buffer 1
   2 μl Enzyme mix B
ii. Run restriction-ligation reaction following Step 2.iii and if not immediately transforming, store at 4° C. for up to one week.
iii. Transform 2 μl of the final restriction-ligation reaction into one aliquot of competent E. coli. Grow on Kanamycin antibiotic resistance, select, and extract plasmid DNA from single white E. coli colonies on red-white selection (no additional reagents necessary).
iv. Verify successful cloning by sequencing with primers A and B that span the ligation sites.
v. Transform 3 μl of plasmid DNA into competent A. tumefaciens. and select on Kanamycin antibiotic resistance with Rifampicin or Gentamycin, depending on the strain.
vi. For plant transformation, follow protocols for A. tumefaciens mediated or biolistic bombardment and select on Hygromycin.

Optionally, plasmid pGMF6 can be used to provide eGFP marker to a final vector if necessary. Also, pGMF5 vector can be substituted with pGMF6 for transient activity in cells

### Example 5. Gene Editing Kit Cloning Components

The gene editing kit of the present disclosure includes six pGMF plasmids. pGMF1, pGMF2, pGMF4, pGMF5, and pGMF6 can be selected on Ampicillin antibiotic resistance and extracting DNA from a single blue colony, if using IPTG/X-gal blue-white selection. pGMF3 is selected on Kanamycin antibiotic resistance and red colonies. Plasmid DNA can be stored at −20° C. Below is a list of the gene editing kit cloning components.
pGMF1: Subcloning vector for gRNA1 Pol III promoter
pGMF2: Subcloning vector for gRNA2 Pol III promoter
pGMF3: Golden Gate Level 2 Backbone
pGMF4: SpCas9
pGMF5: Dicot/Monocot Hygromycin resistance marker
pGMF6: Dicot/Monocot nuclear localized eGFP
Premixed Buffer 1: T4 DNA Ligase Buffer (10×); Bovine Serum Albumin (10×), Double-distilled nuclease-free water
Enzyme Mix A: Type II RE BsaI-HFv2 (20 U/μl); T4 DNA Ligase (400,000 U/μl)
Enzyme Mix B: Type II RE BbsI-HF (20 U/μl); T4 DNA Ligase (400,000 U/μl)
Control gRNA1 annealed primer pair 1 (SEQ ID NOs: 18 and 19)
Control gRNA2 annealed primer pair 2 (SEQ ID NOs: 20 and 21)

Sequencing Primer A:
(SEQ ID NO: 12)
GAACCCTGTGGTTGGCATGCACATAC
Sequencing Primer B
(SEQ ID NO: 13)
AGATAAGGGAATTAGGGTTC

FIGS. 5A and 6A demonstrate an empty pGMF1 subcloning vector for gRNA1 and gRNA2, respectively. The lacZ gene is designed to be replaced with gRNA annealed primer pairs. The promoter for driving expression of gRNA is a rice OsU6-2 RNA polymerase III promoter.

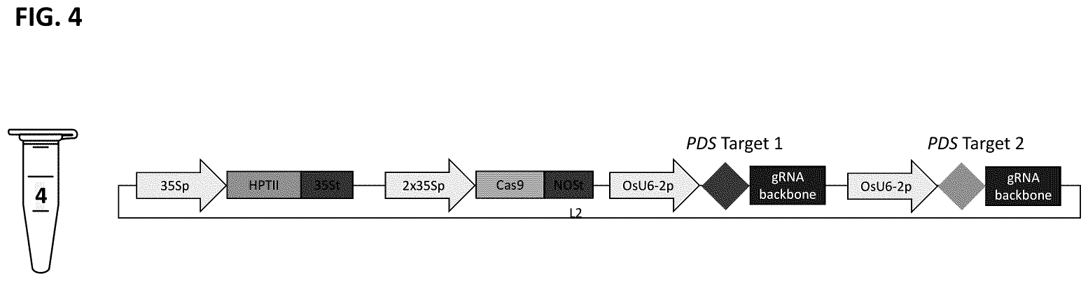

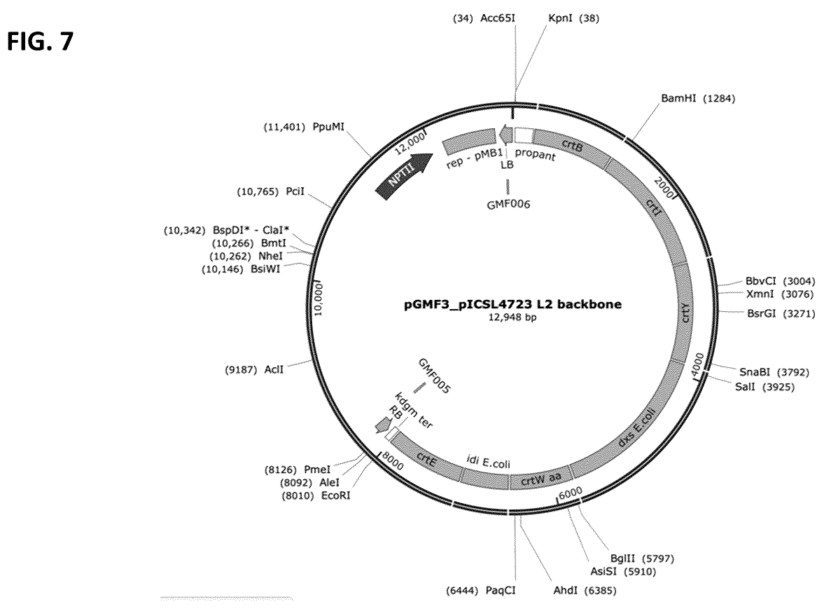

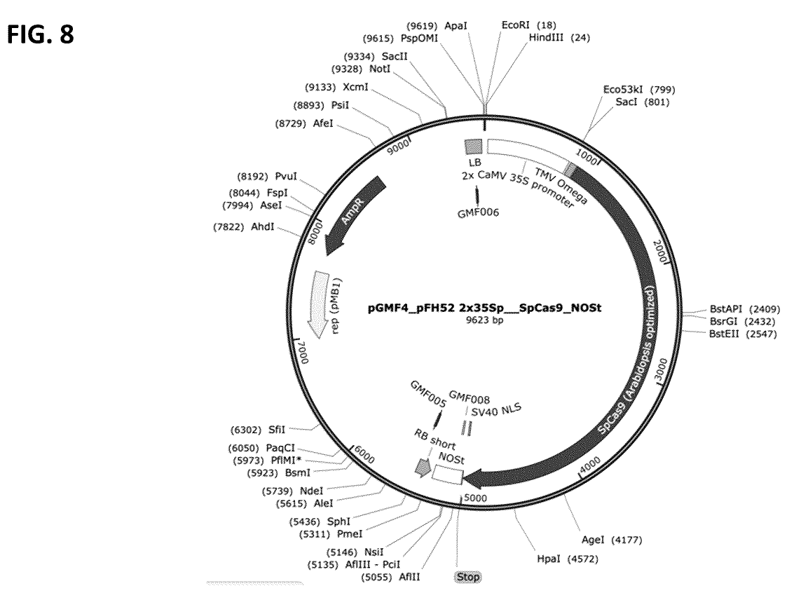

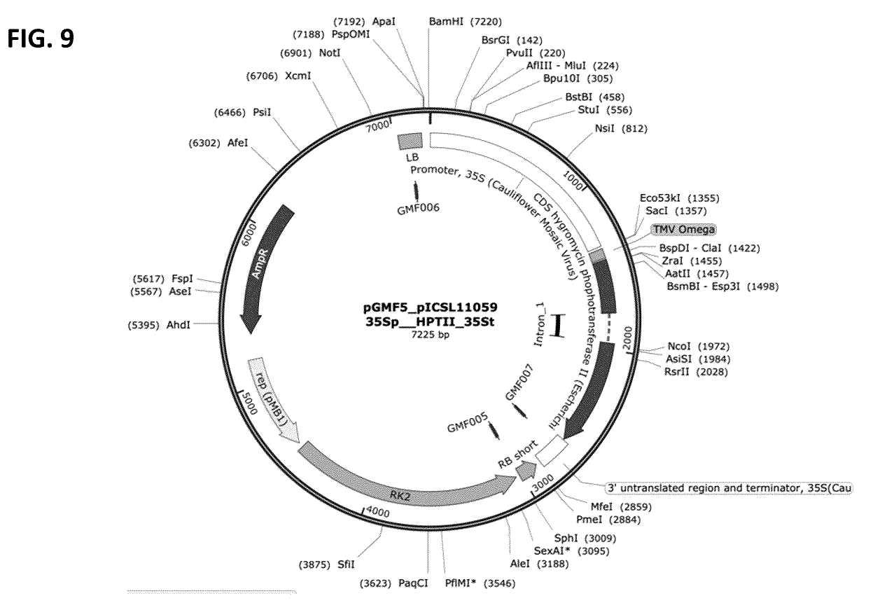

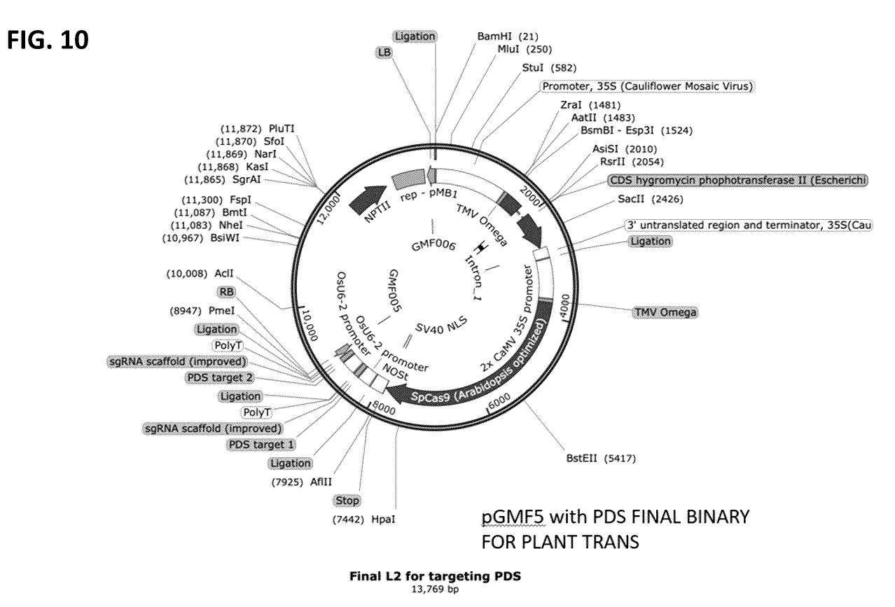

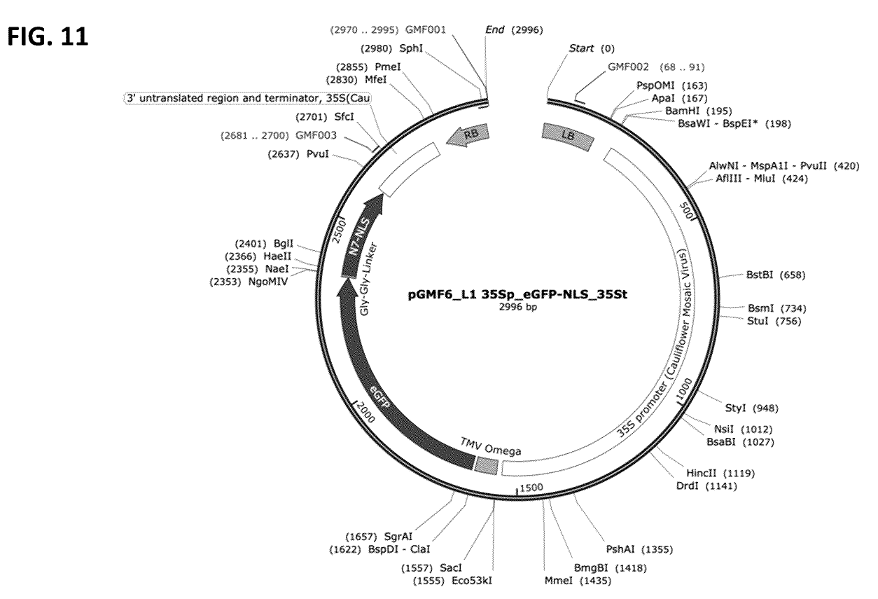

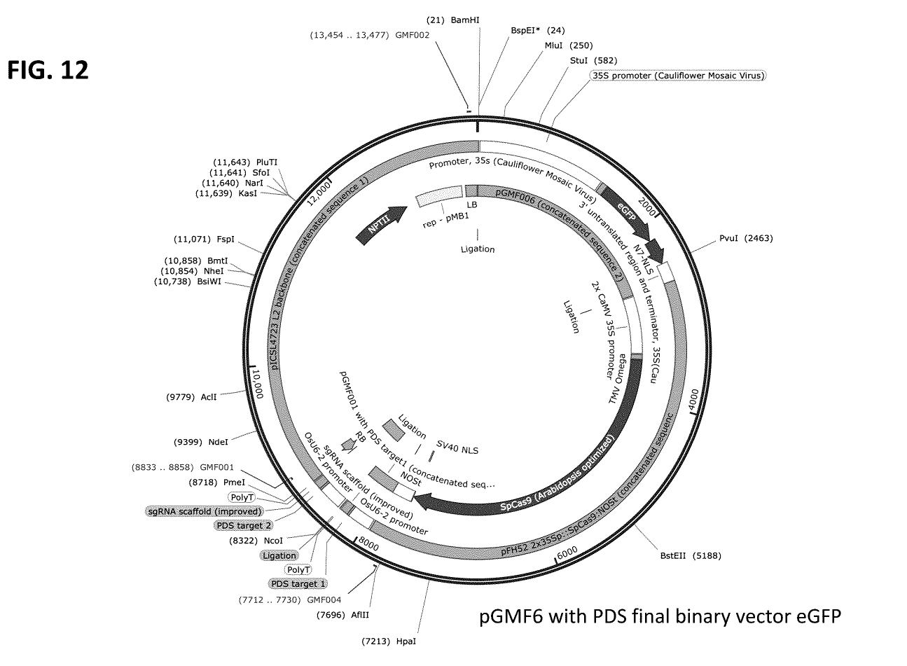

FIGS. 5B and 6B demonstrate a pGMF1 subcloning vector for gRNA1 targeting PDS and gRNA2 targeting PDS, respectively, which are used as a positive control. The promoter for driving gRNA expression is a rice OsU6-2 promoter. The promoter can be used from a rice OsU6-2 promoter, an Arabidopsis AtU6-26 promoter, an RNA polymerase III promoter or any other promoter for its purpose. FIGS. 7, 8, 9, and 11 present pGMF3, pGMF4, pGMF5, and pGMF6 vectors, respectively. FIG. 10 displays a final binary vector constructed for editing PDS gene using the ENABLE gene editing cloning system. Four expression cassettes are present in the final binary vector for expression of two gRNAs targeting PDS driven by the OsU6-2 RNA polymerase III promoter, Cas9 driven by 2×CaMV 35S promoter, and hygromycin phosphotransferase II driven by 35S promoter. FIG. 10 is a map of the control vector illustrated in FIG. 4. FIG. 12 displays a final binary vector constructed for editing PDS gene (as a control) using the gene editing system of the present disclosure. Four cassettes are present in the final binary vector for expression of two gRNAs targeting PDS, Cas9, and eGFP.

### Example 6. Using the Gene Editing Cloning System in Orphan Crops

The gene editing cloning system and kits developed by inventors are used to perform CRISPR-Cas9 system with two guide RNA on target gene editing in plants, including orphan crops. The orphan crop that can be applied with the gene editing cloning system disclosed herein is cassava, cowpea, plantain, millet, sweet potato, sorghum, teff, or yam.

### Further Numbered Embodiments of the Disclosure

Other subject matter contemplated by the present disclosure is set out in the following numbered embodiments:
1. A gene editing cloning system characterized by a plurality of expression cassettes comprising:
   (i) at least two guide RNAs (gRNAs), each of which comprises a guide sequence operably linked to a gRNA scaffold sequence wherein a stop signal is operably linked to the gRNA scaffold sequence;
   (ii) a gene encoding a CRISPR-associated (Cas) protein; and
   (iii) a selectable marker gene;
   wherein each expression cassette comprises a promoter,
   wherein the gene editing cloning system is utilized to edit a genome in a plant, a plant part or a plant cell thereof,
   wherein the gRNA comprises at least 15 nucleotides guide sequence complementary to a target gene sequence,
   wherein the gRNA scaffold sequence is a nucleic acid sequence at least 90% identical to SEQ ID NO: 2.
2. The gene editing cloning system of embodiment 1, wherein the promoter for expression of each gRNA is a rice OsU6-2 RNA polymerase III promoter.
3. The gene editing cloning system of embodiment 1, wherein the promoter for expression of the gene encoding the Cas protein is a 2× 35S Cauliflower mosaic virus (CaMV) promoter.
4. The gene editing cloning system of embodiment 1, wherein the promoter for expression of the selectable marker gene is a 35S Cauliflower mosaic virus (CaMV) promoter.
5. The gene editing cloning system of embodiment 1, wherein the Cas protein is Cas9.
6. The gene editing cloning system of embodiment 1, wherein the gene encoding the Cas protein is plant-optimized.
7. The gene editing cloning system of embodiment 1, wherein the selectable marker gene is hygromycin phosphotransferase (HPTII) gene.
8. The gene editing cloning system of embodiment 1, wherein the stop signal is poly thymine (poly T).
9. The gene editing cloning system of embodiment 1, wherein the gRNA binds to at least one genomic region of a target gene, thereby acquiring or enhancing a trait selected from the group consisting of pest and pathogen resistance, insect resistance, plant-disease resistance, drought tolerance, flood tolerance, nutrient biofortification and high yield.
10. The gene editing cloning system of embodiment 1, wherein the plant is a monocot or a dicot.
11. The gene editing cloning system of embodiment 1, wherein the plant is an orphan crop.
12. The gene editing cloning system of embodiment 11, wherein the orphan crop is cassava, cowpea, plantain, millet, sweet potato, sorghum, teff, or yam.
13. The gene editing cloning system of embodiment 1, wherein the gRNA scaffold sequence is a nucleic acid sequence at least 95%, 98%, or 99% identical to SEQ ID NO: 2.
14. The gene editing cloning system of claim 2, wherein the promoter for expression of each gRNA is SEQ ID NO: 24 or a sequence at least 90% identical thereto.
15. A cloning kit comprising:
   (i) at least two cloning vectors for gRNAs, each of which comprises a cassette comprising a promoter operably linked to a lacZ gene and a gRNA scaffold;
   (ii) a vector comprising a cassette comprising a gene encoding a CRISPR-associated (Cas) protein;
   (iii) a vector comprising a cassette comprising a first selectable marker;
   (iv) a destination vector comprising a second selectable marker;
   Optionally (v) a vector comprising a cassette comprising a third selectable marker, wherein the lacZ gene in each of said two cloning vectors is designed to be replaced with a guide sequence complementary to a target gene sequence in a plant cell by a restriction-ligation reaction,
   wherein each cassette has unique overhangs at 5′ and 3′ ends for orderly assembly of multiple cassettes or fragments;
   wherein multiple cassettes comprising said at least two cassettes from (i), said cassette from (ii), and said cassette from (iii) are assembled into a destination vector based on the unique overhangs, and
   wherein the destination vector comprises the assembled multiple cassettes from (i), (ii), and (iii) vectors.
16. The cloning kit of embodiment 15 further comprising a premixed buffer, a first enzyme mix, a second enzyme mix, and at least one annealed primer pair as a control.
17. The cloning kit of embodiment 15, wherein the gRNA scaffold sequence is a nucleic acid sequence at least 90% identical to SEQ ID NO:2.
18. The cloning kit of embodiment 15, wherein each of said at least two cloning vectors comprises a promoter for expression of each gRNA.
19. The cloning kit of embodiment 18, wherein the promoter is a rice OsU6-2 RNA polymerase III promoter.
20. The cloning kit of embodiment 15, wherein the Cas protein is Cas9.
21. The cloning kit of embodiment 15, wherein the gene encoding the Cas protein is plant-optimized.
22. The cloning kit of embodiment 15, wherein the first selectable marker gene is hygromycin phosphotransferase (HPTII) gene.
23. The cloning kit of embodiment 16, wherein the first enzyme mix comprises BsaI type IIS restriction endonuclease.
24. The cloning kit of embodiment 16, wherein the second enzyme mix comprises BbsI type IIS restriction endonuclease.
25. The cloning kit of embodiment 15, wherein the second selectable marker gene is a red color selectable marker that is designed to be replaced with the assembled multiple cassettes.
26. The cloning kit of embodiment 15, wherein the editing of the target gene confers a trait selected from the group consisting of pest and pathogen resistance, insect resistance, plant-disease resistance, drought tolerance, flood tolerance, nutrient biofortification and high yield.
27. The cloning kit of embodiment 15, wherein the plant cell is derived from a monocot or a dicot.
28. The cloning kit of embodiment 15, wherein the plant cell is derived from an orphan crop.
29. The cloning kit of embodiment 28, wherein the orphan crop is cassava, cowpea, plantain, millet, sweet potato, sorghum, teff, or yam.
30. The cloning kit of embodiment 15, wherein the gRNA scaffold sequence is a nucleic acid sequence at least 95%, at least 98%, or at least 99% identical to SEQ ID NO: 2.
31. The cloning kit of claim 1, wherein the promoter for expression of each gRNA is SEQ ID NO: 24 or a sequence at least 90% identical thereto.
32. The cloning kit of embodiment 16, wherein the third selectable marker gene is GFP or eGFP.
33. A vector comprising the gene editing cloning system of embodiment 1.
34. A plant, a plant part, or a plant cell thereof, comprising the vector of embodiment 33, wherein the target gene is edited and wherein plant confers a trait selected from the group consisting of pest and pathogen resistance, insect resistance, plant-disease resistance, drought tolerance, flood tolerance, nutrient biofortification and high yield.
35. A tissue culture of cells produced from the plant of embodiment 34, wherein the tissue cultured cells comprise the vector.
36. A plant seed produced by growing the plant of embodiment 34, wherein the target gene is edited in the seed.
37. A plant or a plant part thereof, produced by growing the seed of embodiment 36.
38. A method for preparing a cloning vector for expression of at least two gRNAs, the method comprising:
   (a) preparing at least two gRNA primer pairs with an overhang at 5′ end, wherein each primer pair is complementary to anneal a double stranded guide sequence molecule, and wherein 5′ end of each primer has the overhang;
   (b) digesting with an restriction enzyme each vector comprising a promoter operably linked to a lacZ gene which is operably linked to a gRNA scaffold and a stop signal, wherein the lacZ gene is removed by the enzymatic reaction;
   (c) ligating the double stranded guide sequence molecule from (a) with the lacZ gene-depleted vector from (b); and
   (d) obtaining a cloning vector that comprises at least two expression cassettes, each of which comprises the promoter, the guide sequence, the gRNA scaffold, and the stop signal;
   wherein the guide sequence at a 5′ end of each gRNA binds to a target gene.
39. The method of embodiment 38, wherein the 5′ overhang is capable of ligating to BsaI restriction site.
40. The method of embodiment 38, wherein the restriction enzyme is BsaI.
41. The method of embodiment 38, wherein the target gene is edited.
42. The method of embodiment 41, wherein a plant with the target gene edited confers a trait selected from the group consisting of pest and pathogen resistance, insect resistance, plant-disease resistance, drought tolerance, flood tolerance, nutrient biofortification and high yield.
43. The method of embodiment 38, wherein the gRNA scaffold sequence is a nucleic acid sequence at least 90%, at least 95%, at least 98%, or at least 99% identical to SEQ ID NO:2.
44. The method of embodiment 38, wherein stop signal is poly-T sequence.
45. A method for producing a gene-edited plant in which a mutation is introduced into a target gene on a genome and no exogenous gene is incorporated on the genome, the method comprising:
   (a) transforming the vector of embodiment 33 into a plant cell;
   (b) culturing the plant cell obtained in the step (a) and selecting a regenerated plant; and
   (c) selecting a plant in which at least one target gene is edited by a CRISPR/Cas system.
46. The method of embodiment 45, wherein the plant cell is derived from a monocot or a dicot.
47. The method of embodiment 45, wherein the plant cell is derived from an orphan crop.
48. The method of embodiment 45, wherein the orphan crop is cassava, cowpea, plantain, millet, sweet potato, sorghum, teff, or yam.
49. The method of embodiment 45, wherein the vector comprises at least two gRNAs, each of which comprises at least 15 nucleotides guide sequence complementary to at least one genomic region of the target gene or functional derivative thereof.
50. The method of any one of embodiments 45-49, wherein said plant cell has one or more mutations in the genome which results in the reduced or abolished expression of the target gene as compared to said expression in a normal cell that does not have such mutations.
51. The method of any one of embodiments 45-50, wherein the target gene is edited.
52. The method of embodiment 45, wherein a plant with the target gene edited confers a trait selected from the group consisting of pest and pathogen resistance, insect resistance, plant-disease resistance, drought tolerance, flood tolerance, nutrient biofortification and high yield.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

### REFERENCES

Jinek M, Chylinski K, Fonfara I, Hauer M, Doudna. J A, Charpentier E. A programmable dual-RNA-guided DNA endonuclease in adaptive bacterial immunity. Science. 2012; 337:816-21.
Kaya H, Mikami M, Endo A, Endo M, Toki S. Highly specific targeted mutagenesis in plants using Staphylococcus aureus Cas9. Scientific Reports. 2016; 6:srep26871.
Steinert J, Schiml S, Fauser F, Puchta H. Highly efficient heritable plant genome engineering using Cas9 orthologues from Streptococcus thermophilus and Staphylococcus aureus. Plant J. 2015; 84:1295-305.
Wolter F, Klemm J, Puchta H. Efficient in planta gene targeting in Arabidopsis using egg cell-specific expression of the Cas9 nuclease of Staphylococcus aureus. The Plant Journal. 2018; 94:735-46,
Schmidt C, Pacher M, Puchta H. Efficient induction of heritable inversions in plant genomes using the CRISPR/Cas system. The Plant Journal. 2019; 98:577-89.
Chatterjee P, Jakimo N, Jacobson J M. Minimal PAM specificity of a highly similar SpCas9 ortholog. Science Advances. 2018; 4:eaau0766.
Nishimasu H, Shi X, Ishiguro S, Gao L, Hirano 5, Okazaki 5, et al. Engineered CRISPR-Cas9 nuclease with expanded targeting space. Science. 2018; eaas9129.
Hu J H, Miller S M, Geurts M H, Tang W, Chen L, Sun N, et al. Evolved Cas9 variants with broad PAM compatibility and high DNA specificity. Nature. 2018; 556:57-63.
Tang X, Lowder L G, Zhang T, Malzahn A A, Zheng X, Voytas D F, et al, A CRISPR-Cpf1 system for efficient genome editing and transcriptional repression in plants. Nature Plants. 2017; 3:nplants201718.
Endo A, Masafumi M, Kaya H, Told S. Efficient targeted mutagenesis of rice and tobacco genomes using Cpf1 from Francisella novicida. Scientific Reports, 2016; 6:srep38169.
Begemann M B, Gray B N, January E, Singer A, Kesler D C, He Y, et al. Characterization and Validation of a Novel Group of Type V, Class 2 Nucleases for in vivo Genome Editing. bioRxiv. 2017; 192799.
Hahn, F., Korolev, A., Sanjurjo Loures, L. et al. A modular cloning toolkit for genome editing in plants. BMC Plant Biol 20, 179 (2020),
Kor S D, Chowdhury N, Keot A K, Yogendra K, Chikkaputtaiah C, Sudhakar Reddy P. RNA Pol III promoters-key players in precisely targeted plant genome editing. Front Genet. 2023 Jan. 4; 13:989199
Venezia M, Creasey Krainer K M. Current Advancements and Limitations of Gene Editing in Orphan Crops. Front Plant Sci. 2021 Sep. 22; 12:742932.
Ntui V O, Tripathi J N, Tripathi L, Robust CRISPR/Cas9 mediated genome editing tool for banana and plantain (Musa spp.), Current Plant Biology, Volume 21, 2020, 100128, ISSN 2214-6628.

1. A gene editing cloning system characterized by a plurality of expression cassettes comprising:
(i) at least two guide RNAs (gRNAs), each of which comprises a guide sequence operably linked to a gRNA scaffold sequence wherein a stop signal is operably linked to the gRNA scaffold sequence;
(ii) a gene encoding a CRISPR-associated (Cas) protein; and
(iii) a selectable marker gene;
wherein each expression cassette comprises a promoter,
wherein the gene editing cloning system is utilized to edit a genome in a plant, a plant part or a plant cell thereof,
wherein the gRNA comprises at least 15 nucleotides guide sequence complementary to a target gene sequence,
wherein the gRNA scaffold sequence is a nucleic acid sequence comprising SEQ ID NO: 2, or a sequence at least 90% identical thereto.

2. The gene editing cloning system of claim 1, wherein the promoter for expression of each gRNA is a rice OsU6 promoter, a rice OsU3 promoter, an Arabidopsis AtU6-26 promoter, a maize ZmU3 promoter, or a soybean GmU6 promoter.

3. The gene editing cloning system of claim 1, wherein the promoter for expression of the gene encoding the Cas protein is a 2× 35S Cauliflower mosaic virus (CaMV) promoter.

4. The gene editing cloning system of claim 1, wherein the promoter for expression of the selectable marker gene is a 35S Cauliflower mosaic virus (CaMV) promoter.

5. The gene editing cloning system of claim 1, wherein the Cas protein is Cas9.

6. The gene editing cloning system of claim 1, wherein the gene encoding the Cas protein is plant-optimized.

7. The gene editing cloning system of claim 1, wherein the selectable marker gene is hygromycin phosphotransferase (HPTII) gene.

8. The gene editing cloning system of claim 1, wherein the stop signal is poly thymine (poly T).

9. The gene editing cloning system of claim 1, wherein the gRNA binds to at least one genomic region of a target gene, thereby acquiring or enhancing a trait selected from the group consisting of pest and pathogen resistance, insect resistance, plant-disease resistance, drought tolerance, flood tolerance, nutrient biofortification and high yield.

10. The gene editing cloning system of claim 1, wherein the plant is a monocot or a dicot.

11. The gene editing cloning system of claim 1, wherein the plant is an orphan crop.

12. The gene editing cloning system of claim 11, wherein the orphan crop is cassava, cowpea, plantain, millet, sweet potato, sorghum, teff, or yam.

13. The gene editing cloning system of claim 1, wherein the gRNA scaffold sequence is a nucleic acid sequence at least 95%, 98%, or 99% identical to SEQ ID NO: 2.

14. The gene editing cloning system of claim 2, wherein the promoter for expression of each gRNA is SEQ ID NO: 24 or a sequence at least 90% identical thereto.

15. A cloning kit comprising:
(i) at least two cloning vectors for gRNAs, each of which comprises a cassette comprising a lacZ gene and a gRNA scaffold;
(ii) a vector comprising a cassette comprising a gene encoding a CRISPR-associated (Cas) protein;
(iii) a vector comprising a cassette comprising a first selectable marker;
(iv) a destination vector comprising a second selectable marker;
Optionally (v) a vector comprising a cassette comprising a third selectable marker,
wherein the lacZ gene in each of said two cloning vectors is designed to be replaced with a guide sequence complementary to a target gene sequence in a plant cell by a restriction-ligation reaction,
wherein each cassette has unique overhangs at 5′ and 3′ ends for orderly assembly of multiple cassettes or fragments;
wherein multiple cassettes comprising said at least two cassettes from (i), said cassette from (ii), and said cassette from (iii) are assembled into a destination vector based on the unique overhangs, and
wherein the destination vector comprises the assembled multiple cassettes from (i), (ii), and (iii) vectors.

16. The cloning kit of claim 15 further comprising a premixed buffer, a first enzyme mix, a second enzyme mix, and at least one annealed primer pair as a control.

17. The cloning kit of claim 15, wherein the gRNA scaffold sequence is a nucleic acid sequence comprising SEQ ID NO: 2, or a sequence at least 90% identical thereto.

18. The cloning kit of claim 15, wherein each of said at least two cloning vectors comprises a promoter for expression of each gRNA.

19. The cloning kit of claim 18, wherein the promoter is a rice OsU6-2 promoter, a rice OsU3 promoter, an Arabidopsis AtU6-26 promoter, a maize ZmU3 promoter, or a soybean GmU6 promoter.

20. The cloning kit of claim 15, wherein the Cas protein is Cas9.

21. The cloning kit of claim 15, wherein the gene encoding the Cas protein is plant-optimized.

22. The cloning kit of claim 15, wherein the first selectable marker gene is hygromycin phosphotransferase (HPTII) gene.

23. The cloning kit of claim 16, wherein the first enzyme mix comprises BsaI type IIS restriction endonuclease.

24. The cloning kit of claim 16, wherein the second enzyme mix comprises BbsI type IIS restriction endonuclease.

25. The cloning kit of claim 15, wherein the second selectable marker gene is a red color selectable marker that is designed to be replaced with the assembled multiple cassettes.

26. The cloning kit of claim 15, wherein the editing of the target gene confers a trait selected from the group consisting of pest and pathogen resistance, insect resistance, plant-disease resistance, drought tolerance, flood tolerance, nutrient biofortification and high yield.

27. The cloning kit of claim 15, wherein the plant cell is derived from a monocot or a dicot.

28. The cloning kit of claim 15, wherein the plant cell is derived from an orphan crop.

29. The cloning kit of claim 28, wherein the orphan crop is cassava, cowpea, plantain, millet, sweet potato, sorghum, teff, or yam.

30. The cloning kit of claim 15, wherein the gRNA scaffold sequence is a nucleic acid sequence at least 95%, at least 98%, or at least 99% identical to SEQ ID NO:2.

31. The cloning kit of claim 18, wherein the promoter for expression of each gRNA is SEQ ID NO:24 or a sequence at least 90% identical thereto.

32. The cloning kit of claim 16, wherein the third selectable marker gene is GFP or eGFP.

33. A vector comprising the gene editing cloning system of claim 1.

34. A plant, a plant part, or a plant cell thereof, comprising the vector of claim 33, wherein the target gene is edited and wherein plant confers a trait selected from the group consisting of pest and pathogen resistance, insect resistance, plant-disease resistance, drought tolerance, flood tolerance, nutrient biofortification and high yield.

35. A tissue culture of cells produced from the plant of claim 34, wherein the tissue cultured cells comprise the vector.

36. A plant seed produced by growing the plant of claim 34, wherein the target gene is edited in the seed.

37. A plant or a plant part thereof, produced by growing the seed of claim 36.

38. A method for obtaining a cloning vector for expression of at least two gRNAs, the method comprising:
(a) preparing at least two gRNA primer pairs with an overhang at 5′ end, wherein each primer pair is complementary to anneal a double stranded guide sequence molecule, and wherein 5′ end of each primer has the overhang;
(b) digesting with an restriction enzyme each vector comprising a promoter operably linked to a lacZ gene which is operably linked to a gRNA scaffold and a stop signal, wherein the lacZ gene is removed by the enzymatic reaction;
(c) ligating the double stranded guide sequence molecule from (a) with the lacZ gene-depleted vector from (b); and
(d) obtaining a cloning vector that comprises at least two expression cassettes, each of which comprises the promoter, the guide sequence, the gRNA scaffold, and the stop signal;
wherein the guide sequence at a 5′ end of each gRNA binds to a target gene.

39. The method of claim 38, wherein the 5′ overhang is capable of ligating to BsaI restriction site.

40. The method of claim 38, wherein the restriction enzyme is BsaI.

41. The method of claim 38, wherein the target gene is edited.

42. The method of claim 41, wherein a plant with the target gene edited confers a trait selected from the group consisting of pest and pathogen resistance, insect resistance, plant-disease resistance, drought tolerance, flood tolerance, nutrient biofortification and high yield.

43. The method of claim 38, wherein the gRNA scaffold sequence is a nucleic acid sequence comprising SEQ ID NO: 2, or a sequence at least 90% identical thereto.

44. The method of claim 38, wherein stop signal is poly-T sequence.

45. A method for producing a gene-edited plant in which a mutation is introduced into a target gene on a genome and no exogenous gene is incorporated on the genome, the method comprising:
(a) transforming the vector of claim 33 into a plant cell;
(b) culturing the plant cell obtained in the step (a) and selecting a regenerated plant; and
(c) selecting a plant in which at least one target gene is edited by a CRISPR/Cas system.

46. The method of claim 45, wherein the plant cell is derived from a monocot or a dicot.

47. The method of claim 45, wherein the plant cell is derived from an orphan crop.

48. The method of claim 47, wherein the orphan crop is cassava, cowpea, plantain, millet, sweet potato, sorghum, teff, or yam.

49. The method of claim 45, wherein the vector comprises at least two gRNAs, each of which comprises at least 15 nucleotides guide sequence complementary to at least one genomic region of the target gene or functional derivative thereof.

50. The method of claim 45, wherein said plant cell has one or more mutations in the genome which results in the reduced or abolished expression of the target gene as compared to said expression in a normal cell that does not have such mutations.

51. The method of claim 45, wherein the target gene is edited.

52. The method of claim 45, wherein a plant with the target gene edited confers a trait selected from the group consisting of pest and pathogen resistance, insect resistance, plant-disease resistance, drought tolerance, flood tolerance, nutrient biofortification and high yield.


SEQUENCE LISTING (24 sequences)

SEQ ID NO: 1 - DNA, 115 bp, synthetic construct
  source 1..115: mol_type = other DNA
gtatcaatgatcgcttgcaagtttcagagctatgctggaaacagcatagcaagttgaaataaggctagtccgttatcaacttgaaaaagtggcaccgagtcggtgcttttttttt

SEQ ID NO: 2 - DNA, 86 bp, synthetic construct
  source 1..86: mol_type = other DNA
gtttcagagctatgctggaaacagcatagcaagttgaaataaggctagtccgttatcaacttgaaaaagtggcaccgagtcggtgc

SEQ ID NO: 3 - DNA, 58 bp, synthetic construct
  source 1..58: mol_type = other DNA
gcttccatgagtcgtagccgtagcgtaagtgctagtttgtgtaccatcgcgtatggat

SEQ ID NO: 4 - DNA, 58 bp, synthetic construct
  source 1..58: mol_type = other DNA
atccatacgcgatggtacacaaactagcacttacgctacggctacgactcatggaagc

SEQ ID NO: 5 - DNA, 23 bp, synthetic construct
  source 1..23: mol_type = other DNA
agtttgtgtaccatcgcgtatgg

SEQ ID NO: 6 - DNA, 24 bp, synthetic construct
  source 1..24: mol_type = other DNA
gttgagtttgtgtaccatcgcgta

SEQ ID NO: 7 - DNA, 24 bp, synthetic construct
  source 1..24: mol_type = other DNA
aaactacgcgatggtacacaaact

SEQ ID NO: 8 - DNA, 23 bp, synthetic construct
  source 1..23: mol_type = other DNA
tacgctacggctacgactcatgg

SEQ ID NO: 9 - DNA, 23 bp, synthetic construct
  source 1..23: mol_type = other DNA
tacgctacggctacgactcaacc

SEQ ID NO: 10 - DNA, 24 bp, synthetic construct
  source 1..24: mol_type = other DNA
gttgtacgctacggctacgactca

SEQ ID NO: 11 - DNA, 24 bp, synthetic construct
  source 1..24: mol_type = other DNA
aaactgagtcgtagccgtagcgta

SEQ ID NO: 12 - DNA, 26 bp, synthetic construct
  source 1..26: mol_type = other DNA
gaaccctgtggttggcatgcacatac

SEQ ID NO: 13 - DNA, 20 bp, synthetic construct
  source 1..20: mol_type = other DNA
agataagggaattagggttc

SEQ ID NO: 14 - DNA, 1307 bp, synthetic construct
  source 1..1307: mol_type = other DNA
tctgactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggatgcagtgggccccactctgtgaagacaaactagaattcgagctcggagggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgttgtgagaccgcagctggcacgacaggtttgccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgacggtctcagttttagagctagaaatagcaagttaaaataaggctagtccgttatcaacttgaaaaagtggcaccgagtcggtgctttttttttcgctttacttgtcttctgcacgaagtggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccca

SEQ ID NO: 15 - DNA, 1307 bp, synthetic construct
  source 1..1307: mol_type = other DNA
tctgactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggatgcagtgggccccactctgtgaagacaattacgaattcccatggggagggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgttgtgagaccgcagctggcacgacaggtttgccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgacggtctcagttttagagctagaaatagcaagttaaaataaggctagtccgttatcaacttgaaaaagtggcaccgagtcggtgctttttttttcgctttacttgtcttctgcacgaagtggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccca

SEQ ID NO: 16 - DNA, 23 bp, unidentified
  source 1..23: mol_type = unassigned DNA; note = Musa acuminata subsp.
gtatcaatgatcgcttgcaatgg

SEQ ID NO: 17 - DNA, 23 bp, unidentified
  source 1..23: mol_type = unassigned DNA; note = Musa acuminata subsp.
ttttgccagccatgcttggaggg

SEQ ID NO: 18 - DNA, 24 bp, synthetic construct
  source 1..24: mol_type = other DNA
gttggtatcaatgatcgcttgcaa

SEQ ID NO: 19 - DNA, 24 bp, synthetic construct
  source 1..24: mol_type = other DNA
aaacttgcaagcgatcattgatac

SEQ ID NO: 20 - DNA, 24 bp, synthetic construct
  source 1..24: mol_type = other DNA
gttgttttgccagccatgcttgga

SEQ ID NO: 21 - DNA, 24 bp, synthetic construct
  source 1..24: mol_type = other DNA
aaacgtatcaatgatcgcttgcaa

SEQ ID NO: 22 - DNA, 26 bp, synthetic construct
  source 1..26: mol_type = other DNA
gaaccctgtggttggcatgcacatac

SEQ ID NO: 23 - DNA, 24 bp, synthetic construct
  source 1..24: mol_type = other DNA
ctggtggcaggatatattgtggtg

SEQ ID NO: 24 - DNA, 247 bp, Oryza sativa
  source 1..247: mol_type = genomic DNA
ggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgttg